(19) United States
(12) Patent Application Publication
(10) Pub. No.: US 2026/0078537 A1
(43) Pub. Date: Mar. 19, 2026

(54) METHOD OF STABILISING NONWOVEN FIBRES AND REUSABLE MOISTURE ABSORBING ARTICLES

(71) Applicant: RSD Holdings Limited, Auckland (NZ)

(72) Inventor: Frantisek Riha-Scott, Auckland (NZ)

(21) Appl. No.: 19/472,503

(22) PCT Filed: Apr. 5, 2024
(86) PCT No.: PCT/IB2024/053323

§ 371 Date: Oct. 3, 2025

(30) Foreign Application Priority Data
Apr. 6, 2023 (AU) 2023901019

(51) Int. Cl. D04H 13/00; A61F 13/15; A61F 13/49; A61F 13/513; A61F 13/514

(52) U.S. Cl. CPC D04H 13/006; A61F 13/15268; A61F 13/15585; A61F 13/49003; A61F 13/513; A61F 13/51401; A61F 2013/15276; A61F 2013/15878; A61F 2013/1591; A61F 2013/15934; D10B 2201/02; D10B 2201/10; D10B 2321/021; D10B 2321/022; D10B 2509/026

(57) ABSTRACT

A method of stabilising nonwoven fibres is disclosed. A stabilising material 120 is applied to at least a portion or all of the nonwoven material 110 that comprises or is in a form of a plurality of nonwoven fibres so that the stabilising material 120 is applied to at least some or all of the nonwoven fibres. The nonwoven fibres to which the stabilising material 120 is applied are bonded with the stabilising material 120 by adhesive and/or heat bonding, thereby stabilising at least the portion or all of the nonwoven material 110.

The present invention relates to a method of stabilising nonwoven fibres, and reusable moisture absorbing articles.

### BACKGROUND

Disposable absorbent articles, such as those designed for urine incontinence or menstrual fluid absorption, are widely used but pose environmental challenges due to their single-use nature. On the other hand, reusable absorbent products, while more eco-friendly, are complex to manufacture and expensive for consumers in comparison to their disposable counterparts, particularly in the early stages of production.

Around the globe, the absorbent hygiene industry is facing a major challenge due to impending regulatory bans on single-use products containing plastic polymers. Current single-use adult hygiene products contain plastic polymers.

Single-use product manufacturers are investigating the use of natural fibres in their products. However, the natural fibres provide far less surface space for moisture to attach to, so they are not as effective as synthetic fibres in terms of absorbance. One solution might be recycling of the synthetic fibres, that requires collecting used hygiene products from the market which can be extremely difficult, if not impossible.

A further way to potentially comply with the impending regulations would be to create nonwoven products that can be washed and reused. However, so far, the single use product manufacturers have not managed to create a viable reusable alternative because nonwoven fibres are so unstable that they simply fall apart when washed or laundered.

Although, reusable hygiene products do exist, they are not currently economically viable to manufacture at a scale. There are simply too expensive, due to the extensive manual labour required to make them and the prohibitive cost of raw materials (knitted and woven textiles are substantially more expensive than nonwoven materials. Compromising on product performance to reduce manufacturing costs is not desirable as poorly performing products can create more waste and can be more expensing to end-users in a long run.

### OBJECT OF THE INVENTION

It is therefore an object of the present invention to provide a method of stabilising nonwoven fibres which overcomes or at least ameliorates some of the disadvantages described above, or which at least provides a public with a useful choice.

Additionally, or alternatively, it is an object of the present invention to provide a reusable moisture absorbing article that is manufactured by stabilising nonwoven fibres which overcomes or at least ameliorates some of the disadvantages described above, or which at least provides a public with a useful choice.

### BRIEF DESCRIPTION OF THE INVENTION

In a first aspect, the invention resides in a method of stabilising nonwoven fibres, the method comprising the steps of:
providing a nonwoven material comprising or in the form of a plurality of nonwoven fibres;
providing a stabilising material;
applying the stabilising material to at least a portion or all of the nonwoven material so that the stabilising material is applied to at least some or all of the plurality of nonwoven fibres, the stabilising material being a material that is configured to bond the nonwoven fibres by adhesive and/or heat bonding; and
bonding the plurality of nonwoven fibres to which the stabilising material is applied with the stabilising material by adhesive and/or heat bonding.

Preferably, the nonwoven fibres are bonded to form a bonded nonwoven fibre layer comprising at least a portion of nonwoven fibres that are bonded to the extent that the bonded nonwoven fibres do not disintegrate or substantially disintegrate when the layer is washed or laundered at least once or multiple times.

Preferably, the nonwoven material is nonwoven material layer.

Preferably, the nonwoven material is a nonwoven a nonwoven fabric or a nonwoven textile.

Preferably, the heat bonding comprises fusing at least some or all of the nonwoven fibres at a temperature that correlates to a melting point of the fibres that are to be fused and/or a melting point of the stabilising material.

Preferably, the heat bonding occurs at a temperature of between 70 degrees Celsius and 240 degrees Celsius.

Preferably, the heat bonding occurs at a temperature of between 100 degrees Celsius and 240 degrees Celsius.

Preferably, the stabilising material is formed as a stabilising layer.

Preferably, the stabilising layer is a membrane or a film.

Preferably, the stabilising layer has a thickness based on a type of the nonwoven material and a melting point of the nonwoven material.

Preferably, the stabilising layer has a thickness between 0.005 mm and 0.5 mm.

Preferably, the stabilising layer is a breathable layer.

Preferably, the stabilising layer is a full layer.

Preferably, the stabilising layer is a partial layer.

Preferably, the stabilising layer is a framed layer.

Preferably, the stabilising layer is a polyurethane (PU) material layer.

Preferably, the stabilising layer is a polyethylene (PE) material layer.

Preferably, the stabilising layer is a textile adhesive layer.

Preferably, the nonwoven fibres are synthetic polymers, biocomponent fibres, natural fibres, manmade fibres or a combination thereof.

Preferably, the synthetic polymers are selected from polyethylene (PE), polypropylene (PP) or polyethylene terephthalate (PET).

Preferably, the natural fibres are selected from wood, pulp, cotton or bamboo.

Preferably, the manmade fibres are selected from Viscose, Lyocell, polylactic acid (PLA)or cellulose acetate.

Preferably, the bonding occurs in a patterned manner.

Preferably, the stabilising material is an adhesive material.

Preferably, the adhesive material is a glue.

Preferably, the adhesive material is a spray or a roll-on glue.

Preferably, the adhesive material is a polyurethane (PU) adhesive.

Preferably, the adhesive material is a polyethylene (PE) adhesive.

Preferably, the adhesive material is a polylactic acid (PLA) adhesive.

Preferably, the adhesive material is a textile adhesive.

Preferably, an adhesive substance is applied to the stabilising material for the bonding.

Preferably, the adhesive substance is a glue.

Preferably, the glue is a spray or a roll-on glue.

Preferably, the adhesive substance is a polyurethane (PU) adhesive.

Preferably, the adhesive substance is a polyethylene (PE) adhesive.

Preferably, the adhesive substance is a polylactic acid (PLA) adhesive.

Preferably, the adhesive substance is a textile adhesive.

In a second aspect, the invention resides in a reusable moisture absorbing article comprising a plurality of the nonwoven fibres that are stabilised using the method as defined by any one or more of the above or below statements.

Preferably, the reusable moisture absorbing article is of or for use with an undergarment to be positioned adjacent a body of a user and to absorb (preferably, absorb and retain) body moisture from the user.

Preferably the reusable moisture absorbing article is one of the following:
a diaper,
a sanitary napkin,
an incontinence pad,
an incontinence pouch,
a female underwear for incontinence,
a male underwear for incontinence,
a nursing pad,
an underpad (preferably an underpad for a bed, underpad for a seat),
a baby diaper changing pad, or
a baby cot protector.

In a third aspect, the invention resides in a method of constructing a reusable moisture absorbing article of or for use with an undergarment to be positioned adjacent a body of a user and to absorb (preferably, absorb and retain) body moisture from the user, the method comprising:
providing at least one a layer of nonwoven material comprising or in the form of a plurality of non-woven fibres;
applying at least one stabilising material to at least a portion of the at least one layer of the nonwoven material so that the stabilising material is applied to at least some or all of the plurality of nonwoven fibres, the stabilising material being a material that is configured to bond the nonwoven fibres by adhesive and/or heat
bonding and the stabilising material forming a stabilising layer; and bonding the plurality of nonwoven fibres to which the stabilising material is applied with the stabilising material by adhesive and/or heat bonding, thereby forming the reusable moisture absorbing article.

Preferably, the nonwoven fibres are bonded to the extent that the bonded nonwoven fibres do not disintegrate or substantially disintegrate when exposed to washing or laundering at least once or multiple times.

Preferably, the heat bonding comprises or involves fusing at least some or all of the nonwoven fibres at a temperature that correlates to a melting point of the fibres that are to be fused and/or a melting point of the stabilising material.

Preferably, the heat bonding comprises or involves fusing at least some or all of the nonwoven fibres at a temperature that correlates to a melting point of the fibres that are to be fused and/or a melting point of the stabilising material.

Preferably, the heat bonding occurs at a temperature of between 70 degrees Celsius and 240 degrees Celsius.

Preferably, the heat bonding occurs at a temperature of between 100 degrees Celsius and 240 degrees Celsius.

Preferably, the stabilising layer is a membrane or a film.

Preferably, the stabilising layer has a thickness based on a type of the nonwoven material layer and a melting point of the nonwoven material layer.

Preferably, the stabilising layer has a thickness between 0.005 mm and 0.5 mm.

Preferably, the stabilising layer is a breathable layer.

Preferably, the stabilising layer is a full layer.

Preferably, the stabilising layer is a partial layer.

Preferably, the stabilising layer is a framed layer.

Preferably, the stabilising layer is a polyurethane (PU) material layer.

Preferably, the stabilising layer is a polyethylene (PE) material layer.

Preferably, the stabilising layer is a polylactic acid (PLA) material layer.

Preferably, the stabilising layer is a textile adhesive layer.

Preferably, the bonding occurs in a patterned manner.

Preferably, the stabilising material is an adhesive material.

Preferably, the adhesive material is a glue.

Preferably, the adhesive material is a spray or a roll-on glue.

Preferably, the adhesive material is a polyurethane (PU) adhesive.

Preferably, the adhesive material is a polyethylene (PE) adhesive.

Preferably, the adhesive material is a polylactic acid (PLA) adhesive.

Preferably, the adhesive material is a textile adhesive.

Preferably, an adhesive substance is applied to the stabilising material for the bonding.

Preferably, the adhesive substance is a glue.

Preferably, the glue is a spray or a roll-on glue.

Preferably, the adhesive substance is a polyurethane (PU) adhesive.

Preferably, the adhesive substance is a polyethylene (PE) adhesive.

Preferably, the adhesive substance is a polylactic acid (PLA) adhesive.

Preferably, the adhesive substance is a textile adhesive.

Preferably, the nonwoven fibres are synthetic polymers, biocomponent fibres, natural fibres, manmade fibres or a combination thereof.

Preferably, the synthetic polymers are selected from polyethylene (PE), polypropylene (PP), polyethylene terephthalate (PET).

Preferably, the natural fibres are selected from wood, pulp, cotton or bamboo.

Preferably, the manmade fibres are selected from Viscose, Lyocell, polylactic acid (PLA) or cellulose acetate.

Preferably, the article is a urine incontinence absorbing article (urine incontinence pouch).

Preferably, the article is a menstrual fluid article (female hygiene article or menstrual hygiene article).

Preferably, the article is a menstrual fluid article (female hygiene article or menstrual hygiene article) that is configured to be positioned adjacent a person's genital for receiving menstrual fluid from genital area of the person.

Preferably the article is one of the following:
a diaper,
a sanitary napkin,
an incontinence pad,
an incontinence pouch,
a female underwear for incontinence,
a male underwear for incontinence, or
a nursing pad,
an underpad (preferably an underpad for a bed, underpad for a seat),
a baby diaper changing pad, or
a baby cot protector.

In a fourth aspect, the invention resides in a method of constructing a reusable moisture retaining article of or for use with an undergarment to be positioned adjacent a body of a user and to absorb and retain body moisture from the user, the method comprising:
providing a reusable moisture absorbing and retaining insert at least a portion of which is exposed or configured to be exposed to define an inner layer of the article, the inner layer being configured to be located adjacent the user's body and facing towards the user's body for being in bodily contact with the user to directly absorb and retain the body moisture from the user, the insert comprising at least one inner layer of a nonwoven material comprising or in the form of a plurality of non-woven fibres;
providing an inner more layer of a nonwoven material comprising or in the form of a plurality of nonwoven fibres located outwardly of the insert;
providing a stabilising layer formed by a stabilising material, the stabilising material being a material that is configured to bond the nonwoven fibres by adhesive and/or heat bonding;
providing an outer cover that is configured to define an outer layer of the article; and
assembling the inset, the inner more layer, the stabilising layer, the outer cover by:
   positioning the inner more layer, the insert, the stabilising layer and the outer cover so that the insert is located between the outer cover and the stabilising layer, at least a first portion of the stabilising layer is located between the inner more layer and the insert, wherein at least a second portion of the stabilising layer is also configured to bond with the outer cover,
   bonding at least a portion of the inner more layer with the stabilising layer by applying heat and/or adhesive bonding,
   bonding at least a portion of the insert with the stabilising layer by applying the heat and/or adhesive bonding, and
   bonding at least a portion of the outer cover with the second portion of the stabilising layer by applying the heat and/or adhesive bonding, the insert being located between the outer cover and the stabilising layer.

Preferably, the nonwoven fibres are bonded to the extent that the bonded nonwoven fibres do not disintegrate or substantially disintegrate when exposed to washing or laundering at least once or multiple times.

Preferably, the insert comprises at least a first inner layer and a second inner layer, the first inner layer being more proximal to the outer cover than the second inner layer and the second inner layer being more proximal to the inner more layer than the second inner layer, at least the first inner layer comprising or in the form of a plurality of nonwoven fibres.

Preferably, the second inner layer is a quick dry knit textile layer.

Preferably, the second inner layer is a quilt stich to reduce friction.

Preferably, the method comprises bonding at least a portion of the second inner layer with the stabilising layer by applying the heat and/or adhesive bonding.

Preferably, the inner more layer is located outwardly of the second inner layer.

Preferably, the heat bonding comprises or involves fusing at least some or all of the nonwoven fibres at a temperature that correlates to a melting point of the fibres that are to be fused and/or a melting point of the stabilising material.

Preferably, the heat bonding occurs at a temperature of between 70 degrees Celsius and 240 degrees Celsius.

Preferably, the heat bonding occurs at a temperature of between 100 degrees Celsius and 240 degrees Celsius.

Preferably, the stabilising layer is a membrane or a film.

Preferably, the stabilising layer has a thickness based on a type of the nonwoven material layer and a melting point of the nonwoven material layer.

Preferably, the stabilising layer has a thickness between 0.005 mm and 0.5 mm. Preferably, the stabilising layer is a breathable layer.

Preferably, the stabilising layer is a full layer.

Preferably, the stabilising layer is a partial layer.

Preferably, the stabilising layer is a framed layer.

Preferably, the stabilising layer is a polyurethane (PU) material layer.

Preferably, the stabilising layer is a polyethylene (PE) material layer.

Preferably, the stabilising layer is a polylactic acid (PLA) material layer.

Preferably, the stabilising layer is a textile adhesive layer.

Preferably, the bonding occurs in a patterned manner.

Preferably, the stabilising material is an adhesive material.

Preferably, the adhesive material is a glue.

Preferably, the adhesive material is a spray or a roll-on glue.

Preferably, the adhesive material is a polyurethane (PU) adhesive.

Preferably, the adhesive material is a polyethylene (PE) adhesive.

Preferably, the adhesive material is a polylactic acid (PLA) adhesive.

Preferably, the adhesive material is a textile adhesive.

Preferably, an adhesive substance is applied to the stabilising material for the bonding.

Preferably, the adhesive substance is a glue.

Preferably, the glue is a spray or a roll-on glue.

Preferably, the adhesive substance is a polyurethane (PU) adhesive.

Preferably, the adhesive substance is a polyethylene (PE) adhesive.

Preferably, the adhesive substance is a polylactic acid (PLA) adhesive.

Preferably, the adhesive substance is a textile adhesive.

Preferably, the nonwoven fibres are synthetic polymers, biocomponent fibres, natural fibres, manmade fibres or a combination thereof.

Preferably, the synthetic polymers are selected from polyethylene (PE), polypropylene (PP), polyethylene terephthalate (PET).

Preferably, the natural fibres are selected from wood, pulp, cotton or bamboo.

Preferably, the manmade fibres are selected from Viscose, Lyocell, PLA or cellulose acetate.

Preferably, the inner more layer defines a window through which a portion of the second inner layer is exposed or configured to be exposed.

Preferably, the inner more layer and the stabilising layer 240 only covers the perimeter of the second inner layer on its surface that is configured to face towards the user's body.

Preferably, the inner more layer comprises a centre portion extending longitudinally from a top end of the article to a bottom end of the article.

Preferably, the inner more layer comprises a first wing and a second wing each located between the top end of the article and the bottom end of the article.

Preferably, the first wing extends away from the centre portion of the inner more layer in a first direction and the second wing extends from the centre portion of the inner more layer in a second direction that is opposite the first direction.

Preferably, the window is located in the centre portion.

Preferably, the first wing and the second wing of the inner more layer each comprise a fastening member for releasable coupling with the undergarment.

Preferably, the article comprises at least one fastener for releasable coupling with the undergarment.

Preferably, the at least one fastener is a hook and loop fastener.

Preferably, the article is a urine incontinence absorbing article (urine incontinence pouch).

Preferably, the article is a menstrual fluid article (female hygiene article or menstrual hygiene article).

Preferably, the article is a menstrual fluid article (female hygiene article or menstrual hygiene article) that is configured to be positioned adjacent a person's genital for receiving menstrual fluid from genital area of the person.

Preferably the article is one of the following:
a diaper,
a sanitary napkin,
an incontinence pad,
an incontinence pouch,
a female underwear for incontinence,.
a male underwear for incontinence,
a nursing pad,
an underpad (preferably an underpad for a bed, underpad for a seat),
a baby diaper changing pad, or
a baby cot protector.

Preferably, the outer cover is formed or assembled to define the outermost layer of the article.

Preferably, the outer cover 250 is a nonwoven layer.

Preferably, the method further comprises:
providing an outermore layer that is configured to be located between the outer cover and the insert; and
assembling the outermore layer to form part of the article by positioning the outermore layer between the outer cover and the insert or the first inner layer of the insert.

Preferably, the outermore layer is a bonding layer so that the insert (or the first inner layer) and the outer cover are bonded together using the outermore layer.

Preferably, the outermore layer is coextensive with the first inner layer and/or the outer cover.

Preferably, the outermore layer is or comprises a bonding glue.

Preferably, the outermore layer is fused with a waterproof textile.

Preferably, the outermore layer is moisture resistant or leakproof layer, thereby functioning as a leakproof barrier.

Preferably, the method comprises applying a moisture resistant solution to the outermore layer.

Preferably, the outermore layer is coated with a moisture resistant solution.

Preferably, the outermore layer is made from the same material(s) as the stabilising layer 220.

Preferably, the stabilising layer is a primary stabilising layer and the outermore layer is a secondary stabilising layer.

Preferably, the article is configured to be positioned adjacent a pudendal region of the user and to receive, absorb the moisture from the pudendal region of the user.

One or more statements relating to any of the above aspects may equally apply to this aspect.

In a fifth aspect, the invention resides in a reusable moisture absorbing article manufactured using a method as defined any or more of the statements for the first aspect or the third aspect or the fourth aspect.

One or more statements relating to any of the above aspects may equally apply to this aspect.

In a sixth aspect, the present invention resides in a reusable moisture absorbing article, the article comprising:
at least one nonwoven material comprising or in the form of a plurality of non-woven fibres;
at least one stabilising material;
wherein at least one nonwoven layer and at least one stabilising layer are bonded together by applying the stabilising material to at least a portion or all of the nonwoven material so that the stabilising material is applied to at least some or all of the plurality of nonwoven fibres; and
bonding the plurality of nonwoven fibres to which the stabilising material is applied with the stabilising material by adhesive and/or heat bonding.

One or more statements relating to any of the above aspects may equally apply to this aspect.

In a seventh aspect, the present invention resides in an undergarment that comprises or incorporates an article that is constructed using any of the method defined above.

Preferably, the undergarment is an underwear.

Preferably, the undergarment is a male underwear.

Ppreferably, the undergarment is a female underwear.

One or more statements relating to any of the above aspects may equally apply to this aspect.

In the above, unless otherwise noted, one or more statements relating to one aspect may equally apply to another aspect(s).

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprises’ or ‘comprised’ or ‘comprising’ is used in relation to the apparatus or to one or more steps in a method or process.

As used hereinbefore and hereinafter, the term “and/or” means “and” or “or”, or both.

As used hereinbefore and hereinafter, “(s)” following a noun means the plural and/or singular forms of the noun.

As used hereinbefore and hereinafter and unless stated otherwise, the word ‘liquid’ in the context of this specification may include ‘liquid’ but may equally include ‘moisture’.

As used hereinbefore and hereinafter and unless stated otherwise, the word ‘moisture resistant’ in the context of this specification may also include ‘water resistant’, ‘waterproof’ and ‘leakproof’.

As used hereinbefore and hereinafter and unless stated otherwise, the word ‘waterproof’ in the context of this specification may include ‘waterproof’ but may also include something that is impervious body moisture.

As used hereinbefore and hereinafter and unless stated otherwise, the word ‘stabilise’ in the context of this specification means to prevent at least some of the nonwoven fibres from disintegrating or easily disintegrating when washed and/or laundered so that the nonwoven fibres and/or an article comprising such nonwoven fibres can be reused even after washing and/or laundering. This rationale will also be used when the term ‘stabilising’ or ‘stabilises’ is used in relation to the layer(s), fibre(s), product or to one or more steps in a method or process.

As used hereinbefore and hereinafter and unless stated otherwise, the word “moisture” in the context of this specification may include any fluid, liquid and/or moisture discharged from a human body and may also include menstrual fluid/discharges and/or urine.

As used hereinbefore and hereinafter and unless stated otherwise, ‘compressive force’ in the context of this specification may include compressive force but may also compressive force applied together with heat at a suitable temperature for bonding purposes. The same rationale applies to ‘compression force’.

As used hereinbefore and hereinafter and unless stated otherwise, the word ‘retain’ in the context of this specification may mean ‘continue to hold or contain something’ but may also be capable of being released such as by evaporation, squeezing and wringing etc. The word ‘retain’ which is different from ‘absorb’ which in the context of the specification may mean ‘to take and/or draw’ in a liquid from the surface or space around'. Unless otherwise state, this rationale will also apply for the word(s) ‘retaining’ and/or ‘retention’ as used hereinbefore and hereinafter.

When used in claim and unless stated otherwise, the word ‘for’ is to be interpreted to mean only ‘suitable for’, and not for example, specifically ‘adapted’ or ‘configured’ for the purpose that is stated.

It is known, to those with skill in the art of patenting, that the word ‘substantially’ can, in some instances, be used to broaden a term. It should be stated that, in the present application, use of the word ‘substantially’ with a term, to define a (characterizing) feature(s), gets all the benefit (i.e. the benefit of any broadening) afforded by use of the word ‘substantially’, and also includes within its scope the feature(s) being that term exactly, (without broadening). For example, if a feature is described/defined in the present application as being ‘substantially vertical’, then that includes, within its scope, the features being ‘close’ to vertical (in so far as the word ‘substantially’ is deemed to broaden the term ‘vertical’), and also includes within its scope the features being ‘exactly’ vertical.

### BRIEF DESCRIPTION OF THE DRAWINGS

The invention will now be described by way of example only and with reference to the drawings in which:

FIGS. 1A-1D are exploded views showing nonwoven fibres being stabilised according to a method of the present invention.

FIG. 2A is an outer view of the article according to a first example of the present invention is assembled form.

FIG. 2B is an inner view of the article of FIG. 2A in an assembled form.

FIG. 2C is an exploded view of the article of FIG. 2A.

FIG. 3A is an outer view of the article according to a second example of the present invention in assembled form.

FIG. 3B is an inner view of the article of FIG. 3A in an assembled form.

FIG. 3C is an exploded view of the article of FIG. 3A.

FIG. 4A is an outer view of the article according to a third example of the present invention in assembled form.

FIG. 4B is an inner view of the article of FIG. 4A in an assembled form.

FIG. 4C is an exploded view of the article of FIG. 4A.

FIG. 5A is an outer view of the article according to a fourth example of the present invention in assembled form.

FIG. 5B is an inner view of the article of FIG. 5A in an assembled form.

FIG. 5C is an exploded view of the article of FIG. 5A.

FIG. 6A is an inner view of the article according to a fifth example of the present invention in assembled form.

FIG. 6B is an outer view of the article of FIG. 6A in an assembled form.

FIG. 6C is an exploded view of the article of FIG. 6A.

FIG. 7A is an inner view of the article according to a sixth example of the present invention in assembled form.

FIG. 7B is an outer view of the article of FIG. 7A in an assembled form.

FIG. 7C is an exploded view of the article of FIG. 7A.

FIG. 8A shows a front view of the women's' underwear comprising an article according to a seventh example of the present invention.

FIG. 8B shows a rear view of the women's' underwear of FIG. 8A.

FIG. 8C is an inner view of the article according to the seventh example of the present invention in assembled form.

FIG. 8D is an exploded view of the article of FIG. 8A.

FIG. 9A shows a front view of the men's' underwear comprising an article according to an eighth example of the present invention.

FIG. 9B shows a rear view of the men's' underwear of FIG. 9A.

FIG. 9C shows a flattened inner view of the men's' underwear of FIG. 9A.

FIG. 9D is an exploded view of the article of men's underwear and the article of FIG. 9A.

FIG. 10A shows an exploded view of an article according to a ninth example of the present invention.

FIG. 11A shows an exploded view of an article according to a tenth example of the present invention.

FIG. 11B shows an assembled view of the article of FIG. 11A.

### DETAILED DESCRIPTION

The present invention provides a reusable moisture absorbing article that is designed to be washable and preferably printable, non-fraying, and dimensionally stable. The article is made by stabilising the nonwoven fabric material using a stabilising material that is otherwise highly absorbent and has quick wicking and moisture retention abilities. This makes it ideal for absorbing exudate from the human body, such as menstrual fluid and urine incontinence.

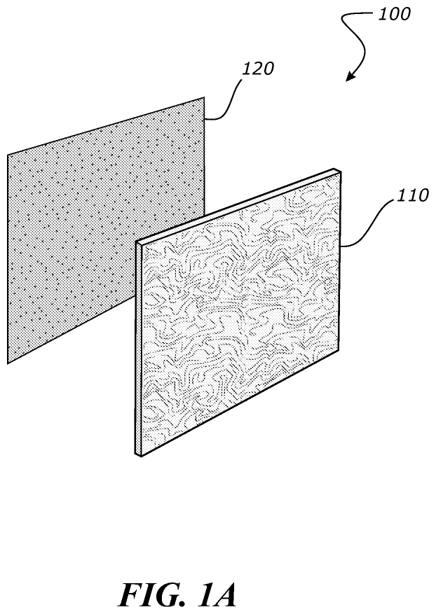

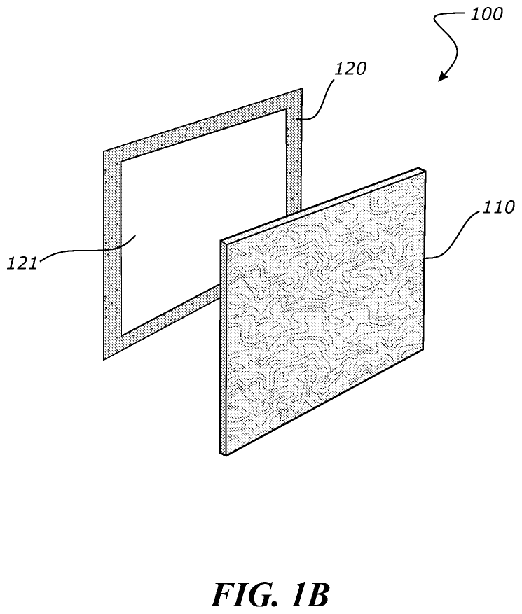

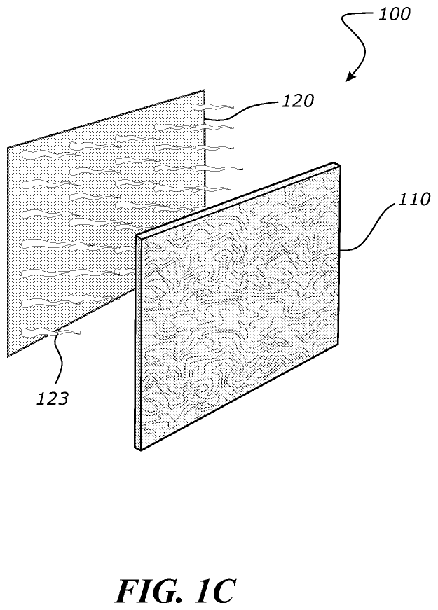

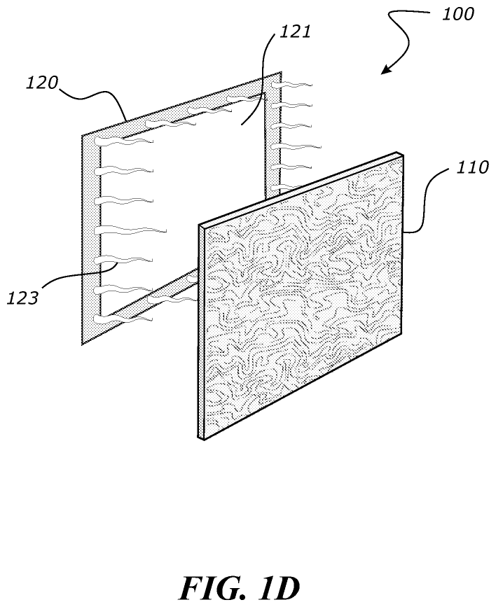

Reference will now be made to the accompanying drawings in which FIGS. 1A-1D show a nonwoven material 110 that is to be stabilised using a stabilising material 120 according to the claimed invention. The nonwoven material 110 comprises or in the form of a plurality of non-woven fibres. The stabilising material 120 is applied to at least a portion or all of the nonwoven material 110. The nonwoven fibres 110 to which the stabilising material 120 is applied are then bonded with the stabilising material 120 by adhesive and/or heat bonding. Such bonding stabilises at least the portion or all of the nonwoven material 110. Nonlimiting examples of possible nonwoven fibres are synthetic polymers such as but not limited to polyethylene (PE), polypropylene (PP), polyethylene terephthalate (PET) etc, biocomponent fibres, natural fibres (e.g. wood pulp, cotton, bamboo etc), manmade fibres (e.g. Viscose, Lyocell, PLA, cellulose acetate) or a combination thereof.

As shown the nonwoven material 110 may be a nonwoven material layer. Hence the same reference numeral 110 has been used to refer to the nonwoven material and the nonwoven layer. The nonwoven material 110 may be a nonwoven a nonwoven fabric or a nonwoven textile.

The process of heat bonding may comprise fusing at least some or all of the nonwoven fibres at a temperature that correlates to a melting point of the fibres that are to be fused/bonded and/or a melting point of the stabilising material. In that way, the fibres and/or stabilising material can melt and that melting can cause the fibres to bond together/fuse to form a nonwoven material of stable construction. Due to the melting of the fibres and fusing of the fibres during heat bonding even those portions of the nonwoven fibres that are not covered by the stabilising layer transform into stable construction. By stable construction, it is meant that the bonded nonwoven fibres will be fused or bonded so as to not disintegrate or easily disintegrate when washed or laundered such that the nonwoven fibres can be reused even after washing or laundering. It is most preferable that the heat bonding occurs at a temperature of between 70 degrees Celsius and 240 degrees Celsius. This temperature range corresponds to the meting point of most if not all possible types of fibres of the nonwoven material 110. In some embodiments, the heat bonding may occur at a temperature range of between 100 degrees Celsius and 240 degrees Celsius.

As shown, the stabilising material 120 may be formed as a stabilising layer. Hence the same reference numeral 120 has been used to refer to the stabilising material and the stabilising layer. The stabilising layer 120 may be a membrane or a film. The stabilising layer 120 may have a thickness based on a type of the nonwoven material and the melting point of the nonwoven material. The stabilising layer 120 may have a thickness between 0.005 mm and 0.5 mm. The stabilising layer 120 may be a breathable layer. The stabilising layer 120 may be a full layer (i.e., fully covering the nonwoven layer) or a partial layer (i.e., fully covering the nonwoven layer) or a framed layer (i.e., having a window portion 121 that does not cover the nonwoven layer). FIG. 1A shows an example where the stabilising layer 120 is a full layer that is configured to fully cover the nonwoven layer 110. FIG. 1B shows an example where the stabilising layer 110 is a framed layer that is configured to covers the nonwoven layer save at the window portion 121. In this example of FIG. 1B, the stabilising layer 121 covers the nonwoven layer at or near the perimeter of the nonwoven layer 110. The stabilising layer 120 may be a polyurethane (PU) material layer. However, it is possible that the stabilising layer 120 may be a polyethylene (PE) material layer, a polylactic acid (PLA) material layer, or many other suitable textile adhesive material layer.

The bonding may occur in a patterned manner.

The nonwoven layer 110 and/or the stabilising layer 120 may be of any suitable shape or size.

The stabilising material may be an adhesive material, preferably a glue (e.g., a spray or a roll-on glue) or a polyurethane (PU) adhesive. However, it is possible that the stabilising material may be a polyethylene (PE) adhesive, a polylactic acid (PLA) adhesive or many other suitable textile adhesive.

In one example, where the bonding is by adhesive bonding only, the nonwoven fibre material 110 may be stabilised by adhesively bonding to the stabilising material using a spray or rolled on adhesive glue.

Similarly, in one example, where the bonding is by heat bonding only, polyurethane (PU) adhesive 123 or similar (e.g., a polyethylene (PE) adhesive, a polylactic acid (PLA) adhesive, or other suitable textile adhesive material layer) may be applied to the stabilising layer 120 to fuse to the nonwoven layer 110 to fuse by heat to the nonwoven layer 110. More specifically, the nonwoven fibres of the nonwoven layer 120 may be stabilised by fusing the nonwoven fibres to a full, partial or framed breathable stabilising material 120 using heat bonding (thermo bonding) at high temperatures ranging from 70 degrees Celsius to 240 degrees Celsius. The polyurethane (PU) films may be of various thickness, preferably ranging from 0.005 mm to 0.5 mm, depending on the type if nonwoven and the melting point of the fibres of the nonwoven material 110. Instead of polyurethane films, it is possible to use polyethylene (PE) films, polylactic acid (PLA) films, or other suitable textile adhesive material films.

Use of polyurethane (PU), polyethylene (PE) or polylactic acid (PLA) in stabilising layer 120 and/or as adhesive is advantageous as that allows manufacturing of sanitary/hygiene products comprising a nonwoven material to be is washable and reusable. Similarly, it allows to manufacture sanitary/hygiene products comprising any number or combination of nonwoven material and knitted or woven material to be is washable and reusable. Many other suitable textile adhesives or material providing same or similar functionality may be used in stabilising layer 120 providing same or similar advantages as polyurethane (PU), polyethylene (PE) or polylactic acid (PLA).

Adding a knitted or woven layer is optional but can be advantageous to further increase and control the stability of nonwoven fibres, allowing for enhanced repeated use or laundering or washing before the nonwoven fibres disintegrate.

The above-described method of the present invention can be used to construct/manufacture a reusable moisture absorbing article of or for use with an undergarment to be positioned adjacent a body of a user and to absorb (and preferably also and retain) body moisture from the user. Some non-limiting examples of such article are a diaper, a sanitary napkin, an incontinence pad, an incontinence pouch, a female underwear for incontinence, a male underwear for incontinence, a nursing pad etc. Therefore, the reusable moisture retaining article may be a reusable sanitary article.

Such article may comprise an inner layer which may be same as nonwoven material 110 described above and the stabilising material 120 as described above forming a stabilising layer. The stabilising material 120 may be applied to at least a portion or all of the inner layer so that the stabilising material is applied to at least some or all of the nonwoven fibres.

The nonwoven fibres 110 to which the stabilising material 120 is applied is then bonded with the stabilising material 120 by adhesive and/or heat bonding, thereby stabilising at least the portion or all of the nonwoven material and forming the reusable moisture absorbing article 100.

In some embodiments, the stabilising material/layer 120 is moisture resistant or leak proof.

### Example 1

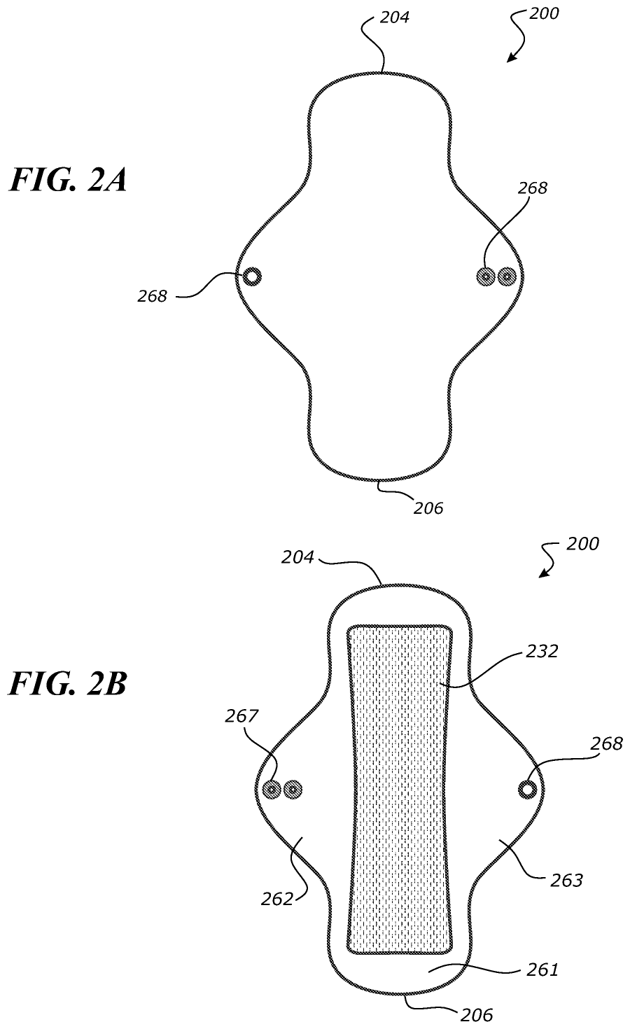

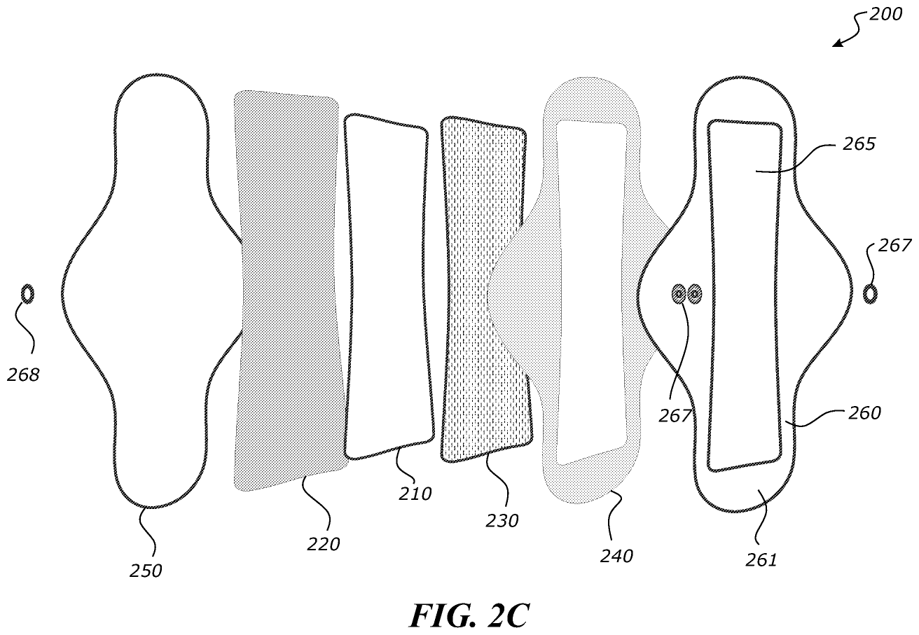

One example of a reusable article is shown in FIGS. 2A-2C. FIG. 2A shows an outer view of a reusable moisture absorbing article 200 according to the present invention. FIG. 2B shows an inner view of the article of FIG. 2A. FIG. 2C shows an exploded view of the article of FIG. 2A and FIG. 2B.

Article 200 is of or for use with an undergarment to be positioned adjacent a body of a user and to receive moisture from the body of the user. The user may be a person wearing the article. The article 200 may be used as a menstrual fluid absorbing article (also referred to as a “female hygiene article” or “menstrual hygiene article, “sanitary napkin”, “female liner” or “menstrual pad”). The article 200 may also be suitable for absorbing any other moisture from the body of the user. The article 200 may also be suitable for absorbing moisture during light to moderate incontinence so can be used as incontinence pad.

The article 200 comprises a nonwoven layer in the form of an inner layer 210 (first inner layer 210) for moisture absorption. The nonwoven layer may be same as the nonwoven layer 110 as described above and therefore need not be described again in detail.

The article 200 comprises a second inner layer 230 and a stabilising layer 240. The stabilising layer 240 may be same or similar to the stabilising layer 120 or stabilising material 120 as described above and therefore need not be explained again in detail. As shown in FIG. 2B at least a portion 232 of the second inner layer 230 is exposed to define an inner region of the article 200. The inner region of the article is configured to be located adjacent the user's body and facing towards the user's body for being in bodily contact with the user to directly receive the moisture from the body of the user.

The portion 232 may optionally be covered by a removable material e.g., removable film or similar that when such removable material is removed, the portion 232 is exposed. This helps prevent the contamination of the portion 232, which is the inner layer of the article that is configured to be in bodily contact with the user.

The article 200 comprises multiple layers as shown in FIG. 2C. These multi-layers are at least an outermost layer 250 (which may also be referred to as an outer cover 250), an outermore layer 220 (which may also be referred to as an outer more layer 220), an insert comprising a first inner layer 210, a second inner layer 230, and a stabilising layer 240. There is also an inner more layer 260 (which may also be referred to as an innermore layer 260). These layers are assembled together to form the article 200. As mentioned above, the first inner layer 210 is a nonwoven layer (preferably same as the nonwoven layer 110 as described above). It functions as an inner nonwoven core of the article 200. The thickness (gsm) of this first inner layer 210 may depend on absorbency levels that is required.

When in the layers are assembled, at least a portion 232 of the second inner layer is exposed or configured to be exposed to define an inner layer of an article. The inner layer (i.e., the portion that is exposed or configured to be exposed) is configured to be located adjacent the user's body to be in bodily contact with the user to directly absorb the moisture from the body of the user. If the article 200 is used as a menstrual fluid and/or urine absorbing article, the moisture of the body of the user that is absorbed through the exposed portion 232 of the insert 232 will be menstrual fluid/menstrual discharge and/or urine.

At least some of the absorbed moisture from the second inner layer 230 may be transferred to the first inner layer 210. By having at least two inner layers 210, 230, the longevity of the article 200 is enhanced. More specifically, having the second inner layer 230 can increase and control the stability of the nonwoven fibres of the first inner layer 210, allowing for more repeated use or laundering before the nonwoven fibres gradually disintegrate. Having two inner layers thereby helps improving the longevity of the article 200. Optionally, there may be more than two inner layers.

As shown in FIG. 2C, the inner more layer 260 may be located outwardly of the second inner layer 230. At least a portion of the stabilising layer 240 may be positioned or sandwiched between the second inner layer 230 and the and the inner more layer 260. More specifically, the, at least a portion of the stabilising layer 240 may be positioned or sandwiched between a surface of the second inner layer 230 that is configured to face towards the inner more layer 260 and a surface of the inner more layer 260 that is configured to face towards the second inner layer 230 thereby interconnecting the second inner layer 230 and the inner more layer 260.

The stabilising layer 240 may be same or similar to the stabilising layer 120 described above both in terms construction material and functionality.

The inner more layer 260 may be the inner most layer of the article 200. The inner more layer 260 may be a nonwoven layer or a nonwoven textile. Hence, in terms of construction, the inner more layer 260 may be similar to the nonwoven layer 110 as described above. The nonwoven fibres of the inner more layer 260 may be stabilised using the stabilising layer 240 in the same or similar manner as described above with reference to FIGS. 1A-1D. The inner more layer 260 may be of the same shape and size as the stabilising layer 240.

The second inner layer 230 and the inner more layer 260 can be bonded together (e.g., by adhesive and/or heat bonding) A compression force (i.e., compressive force) may be applied to the article 200 to facilitate such bonding. This eliminates or at least reduces stitching and/or other complex process for bonding the layers together thereby significantly reducing the manufacturing process and costs. Also, with such a bonding technique complex machinery will note be required to manufacture the article 200.

Reverting to the embodiment of FIG. 2C, the inner more layer 260 may define a window 265 through which the portion 232 of second inner layer 230 is exposed or configured to be exposed. As shown, a substantial portion of the second inner layer is exposed or configured to be exposed to form the inner layer of the article 200. In this example shown, all of the inner layer 230 is exposed or configured to be exposed to form the inner layer of the article 200 except its edges. Hence, on its surface that is configured to face towards the user's body, only the edges or perimeter of the second inner layer 230 may be unexposed or configured to be unexposed. The inner more layer 260 and the stabilising layer 240 may only cover the perimeter of the second inner layer 230 on its surface that is configured to face towards the user's body. If the second inner layer 230 is nonwoven, the nonwoven fibres of the second inner layer 230 may still be stabilised using the stabilising layer 240 and by adhesive and/or heat bonding in a manner as described above with reference to FIGS. 1A-1D.

The inner more layer 260 may comprise a centre portion 261 extending longitudinally from a top end 204 of the article 200 to a bottom end 206 of the article. The inner more layer 260 may comprise a first wing 262 and a second wing 263 each located between the top end 204 and the bottom end 206 of the article 200. The first wing 262 may extend away from the centre portion 261 of the inner more layer 260 in a first direction and the second wing 263 may extend from the centre portion 261 of the inner more layer 260 in a second direction that is opposite the first direction. The window 265 may be located in the centre portion.

In a possible embodiment where no second inner layer 230 is present, the stabilising layer 240 may be sandwiched directly between the first inner layer 210 and the inner more layer 260 in a same or similar manner as it would have otherwise sandwiched between the second inner layer 230 and the inner more layer 260. In such a case, a portion of the first inner layer 210 would be exposed in a similar manner how the portion 232 of the second inner layer is exposed, thereby the exposed portion of the first inner layer being the inner most layer of the article 200. In such a case, the nonwoven fibres of the first inner layer 210 may still be stabilised using the stabilising layer 240 and by adhesive and/or heat bonding in a manner that is same as or similar as described above with reference to FIGS. 1A-1D.

The outer cover 250 is formed or assembled to define the outermost layer of the article.

The outermore layer 220 is positioned or sandwiched between the first inner layer 210 and the outer cover 250 thereby interconnecting the first inner layer 210 and the outer cover 250. More specifically, the outermore layer 220 is positioned or sandwiched between a surface of the first inner layer 210 that is configured to face towards the outer cover 250 and a surface of the outer cover 250 that is configured to face towards the first inner layer thereby interconnecting the first inner layer 210 and the outer cover 250.

The stabilising layer 240 may bond with at least the second inner layer 240, the inner more layer 260 and the outer cover 250.

In this example, since the first inner layer 210 is sandwiched between other layers as shown in FIG. 2C, the fibres of that nonwoven layer cannot easily disintegrate during washing and/or laundering. It is possible that the stabilising layer 240 also bonds with all the layers. For example, outermore layer 220, first inner layer 210 and second inner layer 230 may be of different suitable sizes or shapes to ensure that the stabilising layer 240 bonds with one or more or all of those layers.

In some embodiments, the outermore layer 220 may be a bonding layer so that the first inner layer 210 and the outer cover 250 are bonded together using the outermore layer 220. The first inner layer 210 and the outer cover 250 can be bonded together by adhesive and/or heat bonding. A compression force (i.e., compressive force) may be applied to facilitate such bonding. This eliminates or at least reduces stitching and/or other complex process for bonding the layers together thereby significantly reducing the manufacturing process and costs. Also, with such a bonding technique, complex machinery will note be required to manufacture the article 200.

The outermore layer 220 may be coextensive with the first inner layer 210 and/or the outer cover 250. The outermore layer 220 may be or may comprise a bonding glue. In some embodiments, the outermore layer 220 may be fused with a waterproof textile. The outermore layer 220 may be moisture resistant or leakproof layer, thereby functioning as a leakproof barrier. In some embodiments, a moisture resistant solution is applied to the outermore layer 220. The outermore layer 220 may be coated with a moisture resistant solution. By serving as a leakproof or moisture resistant barrier, the outermore layer provides the leakproof properties of the article 200 thereby providing ultimate protection to the user. More specifically, this helps prevent any absorbed moisture in the first inner layer 210 from leaking to the outer cover 250.

In some embodiments, the outermore layer 220 may also be made from the same materials as the stabilising layer 240, in which case the stabilising layer 240 will be a primary stabilising layer and the outermore layer 220 will be secondary stabilising layer. Since the first inner layer 210 is nonwoven layer, such secondary stabilising layer can further stabilise the nonwoven fibres of the first inner layer 210. Such further stabilisation may occur in the same or similar manner as described above with reference to FIGS. 1A-1D.

The article 200 may be configured to be positioned adjacent a pudendal region of the user and to receive, absorb the moisture from the pudendal region of the user. The article 200 may be or also be used for receiving or absorbing the urine incontinence. The received, absorbed moisture may optionally be retained by the article 200.

The outer cover 250 is most preferably a nonwoven layer. In such a case, the nonwoven fibres of the outer cover 250 may still be stabilised by adhesive and/or heat bonding in a manner as described above with reference to FIGS. 1A-1D. A portion of the stabilising layer 240 that is configured to bond with the outermost layer may be used to stabilise at least the portion of the outer cover in the same or similar manner as described above with reference to FIGS. 1A-1D. Similarly, if the outermore layer 220 is a secondary/second stabilising layer (as described above) then in that case, the nonwoven fibres of the first inner layer 210 and the outer layer 250 will be further stabilised by that outermore layer 220 in the same or similar manner as described above with reference to FIGS. 1A-1D. Melting of the nonwoven fibres of the outer cover 250 during heat bonding may also cause the stabilisation of the fibres of the outer cover in the same or similar way as already described above with reference to FIGS. 1A-1D.

As already explained, the first inner layer 210 is a nonwoven layer. However, the second inner layer 230 may be nonwoven, woven or knitted moisture wicking layer. In some embodiments the second inner layer 230 may be a moisture retention textile assembly as described in PCT Publication number: WO2020121176A1, the entirety of which is herein incorporated by reference.

The first wing 262 and the second wing 263 of the inner more layer 260 may each comprise a fastener(s) 267 (e.g., buttons/snap domes/poppers hook and loop fasteners or Velcro® fasteners) for releasable coupling with the undergarment such as an underwear. For example, the fasteners in the first wing and the second wing may be coupled there by forming loop around the crotch region of the underwear and releasably coupling with the underwear. Alternatively, the undergarment may have a fastener(s) suitable to be coupled to fastener(s) 267.

The outer cover 250 and the inner more layer 260 may be identical in shape and/or size, except that centre portion of the inner more layer 260 comprises a window 265 whereas no such window appears in the outer cover 250. Therefore, the outer cover 250 may comprise a centre portion 251 extending longitudinally from a top end 204 of the article to a bottom end 206 of the article 200. The outer cover 250 may comprise a first wing 252 and a second wing 253 each located between the top end 204 the bottom end 206 of the article 200. The first wing 252 may extend away from the centre portion 251 of the outer cover 250 in a first direction and the second wing 253 may extend from the centre portion 251 of the outer layer 250 in a second direction that is opposite the first direction.

Additionally, or alternatively, the outer cover 250 may comprise similar fastening members 268 (e.g., buttons/snap domes/poppers/hook and loop fasteners or Velcro® fasteners) for releasable coupling with the undergarment such as an underwear. Where both the outer cover and the inner more layer comprise the fasteners, the fasteners 267 and 268 may be suitable to be coupled together. Alternatively, the undergarment may have a fastener(s) suitable to be coupled to fastener(s) 268.

The outer cover 250 and the inner more layer 260 may extend longitudinally from a top end 204 of the article 200 to a bottom end 206 of the article 200. The first and second inner layers 210, 230 and the outermore layer 220 may each be of the smaller than the centre portion 251 of the outer cover 250 and/or the centre portion 261 of the inner more layer 260. In some embodiments, the first and second inner layers 210, 230 and the outermore layer 220 may each be of the same size as the centre portion 251 of the outer cover 250 and/or the centre portion 261 of the inner more layer 260. In some embodiments, the outermore layer 220 is larger (preferably slightly larger) than first and second inner layers 210 and 230. Such configurations can allow the stabilising layer 240 to seal and/or bond or at least be in direct contact with the outermore layer. Different dimensions/sizes and/or shapes of layers are possible to allow the stabilising layer to be in direct contact and/or seal and/or bond with respective layers. In some embodiments, all layers 250, 220, 210, 230, 240 and 260 may extend longitudinally from the top end 204 of the article 200 to a bottom end 206 of the article 200.

In one embodiment, the article 200 may be of elongate shape with each of the layers extending longitudinally the top end 204 and the bottom end 206 and without any wing. Such article may optionally comprise a strap or similar fastening means for releasable coupling with the undergarment.

In one embodiment, the outer cover 250 may be or may also be optional. However, having the outer cover is most preferable due to advantageous features of the outer cover 250 as already explained above.

The adhesive and/or heat bonding of layers of the article 200 may be using same method as described above with reference to FIGS. 1A-1D. Similarly, the fibres of the nonwoven layer(s) of the article 200 are stabilised by the stabilising layers in the same manner as already described above and also with reference to FIGS. 1A-1D and therefore need not be described again.

### Example 2

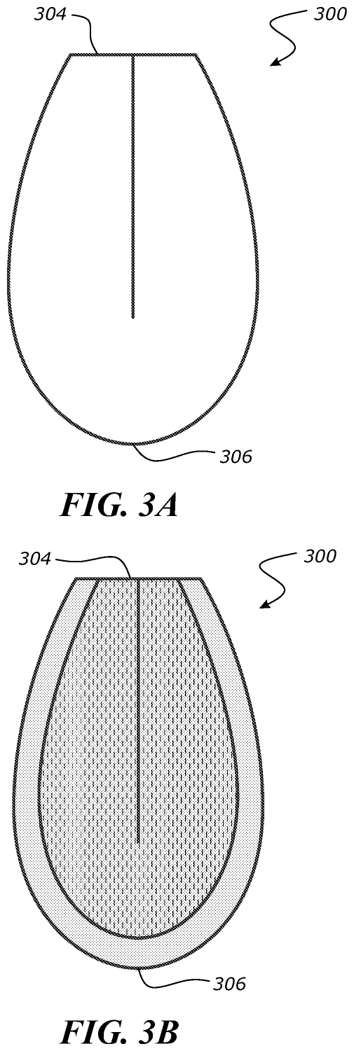

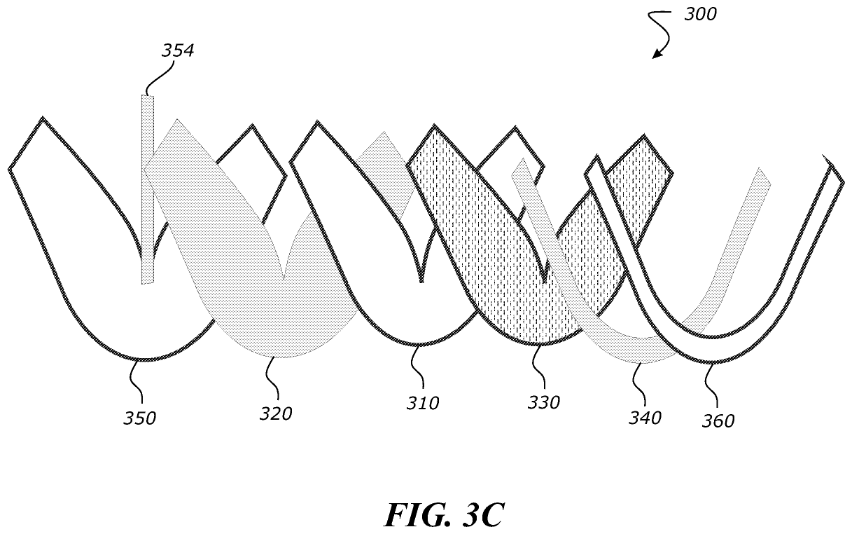

FIGS. 3A-3C show an article 300 according to another preferred embodiment of the present invention.

The article 300 is of or for use with an undergarment to be positioned adjacent a body of a user and to receive moisture from the body of the user. The user may be a person wearing the article. The article 300 may be used as a urine incontinence absorbing and inserting article (also referred to as a “male shield” or “male guard”, or “male pouch” or incontinence pouch”). The article 300 may also be suitable for absorbing any other moisture from the body of the user. In some embodiments, the article is an incontinence article for female that is configured to be positioned adjacent a person's genital for receiving menstrual or other fluid from genital area of the person, including urine.

The article 300 of this example is similar in most aspects to the article 200 described above and the differences can be identified by comparing FIG. 2A with FIG. 3A, FIG. 2B with FIG. 3B, and FIG. 2C with FIG. 3C. In figures relating to the article 300, the features that are similar to those shown in figures relating to article 200 are identified with the same reference numeral, incremented by 100. Most of the description of the article 200 of a preferred embodiment above, equally applies to the article 300 and therefore need not be described again in too much detail. Mostly, only the main differences will be discussed.

One main difference is in the shape of the layers all of which are suitable shaped to function as the urine incontinence absorbing and inserting article.

The inner more layer 360 is the form of a strip. A stabilising layer 340 is also in the form of a strip. The stabilising layer 340 may be an adhesive frame or a stabilised adhesive frame. The stabilising layer 340 may be positioned or sandwiched between the second inner layer 330 and the inner more layer 360. Similarly, the outermore layer 320 may be positioned or sandwiched between the first inner layer 310 and the outer cover 350.

The inner more layer 360 may be made from the same textiles as the outer cover 350. Inner more layer and/or the outer cover 350 may be made from nonwoven textiles or nonwoven material having a plurality of nonwoven fibres. Such nonwoven fibres are stabilised using adhesive and/or heat bonding in a manner that is same or similar to the manner as already described above.

The first and second inner layers 310, 330 may be of same or similar type as already described above for article 200. Similarly, the outermore layer 320 may also be a stabilising layer (secondary stabilising layer) similar to that in Example 1. Any layers that are described as being optional in article 200 above may also be optional in article 300.

In some embodiment the outer cover 350 may already come optionally with stitches that are provided to allow the outer cover 350 to be of a particular shape. In such case, a stitch bonding adhesive layer 354 may optionally be provided to extend along and conceal such stitches for extra protection. The stitch bonding adhesive layer 354 may be a stitch binding glue seal or a strip and may be or may function as a thermo fused adhesive strip. The stitch bonding adhesive layer 354 may be made from the same material as the first stabilising layer 320. It is to be noted that the stitches may not be present in the outer cover 350 and alternative methods to stitches (e.g., creases). may be used to create the shape of the article 300. But even if present they are pre-formed stitches and the stitches that is configured to bond multiple layers together. The multiple layers may bonded by applying adhesive and/or heat bonding, preferably applying compressive force as described above. Thermo melt or ultrasonic melt seam is more preferable to stiches.

Other than the above-mentioned differences and the differences that are obvious from the figures, all other features of the article 300 including the properties of its various layers may be same as that described above with reference to article 200.

### Example 3

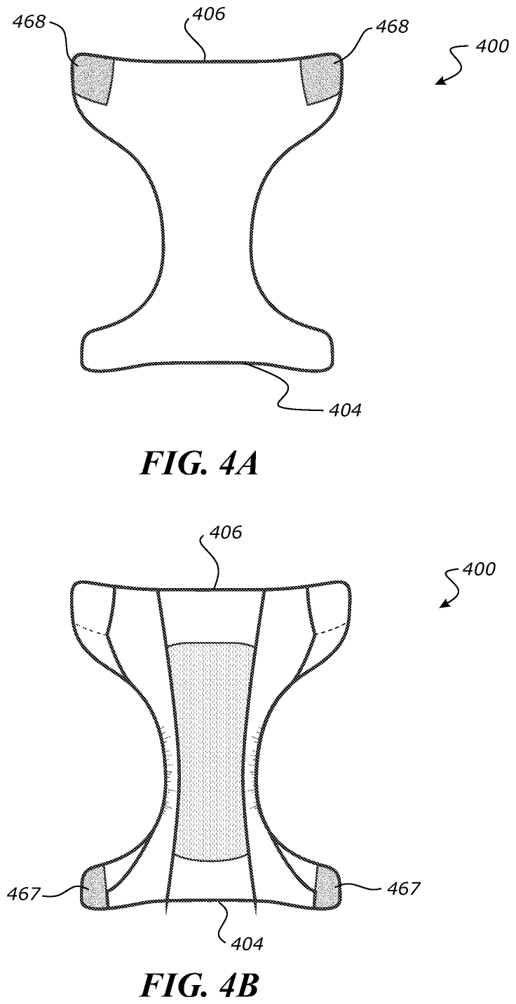

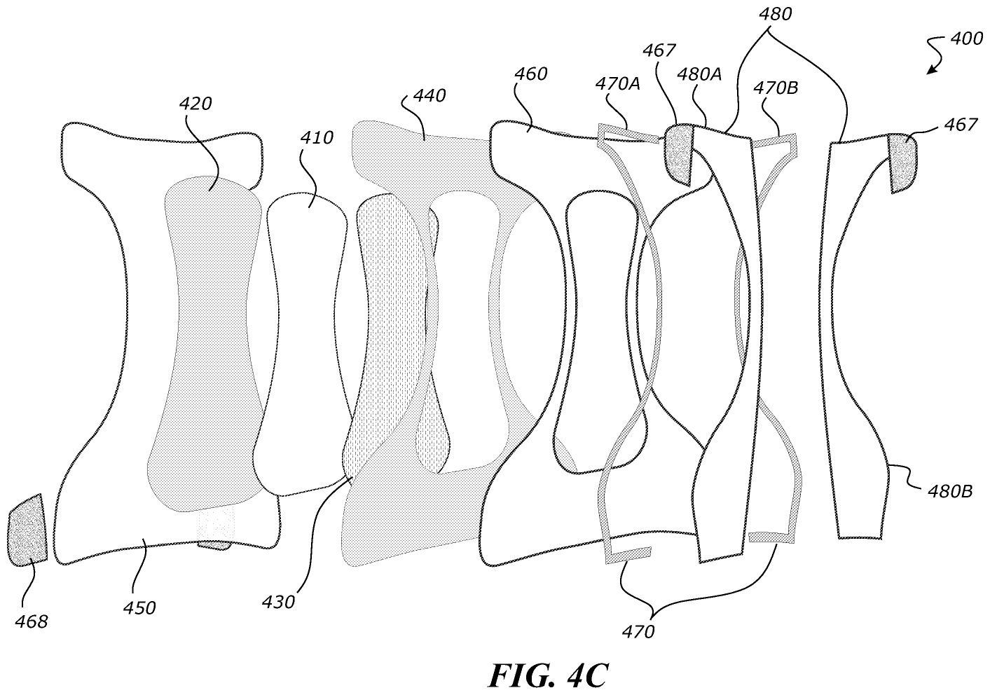

FIGS. 4A-4C show an article 400 according to another preferred embodiment of the present invention. The article 400 in this example is a baby diaper or training pants.

The article 400 is of or for use with an undergarment to be positioned adjacent a body of a user and to receive moisture from the body of the user. The user may be a person wearing the article.

The article 400 of this example is similar in most aspects to the article 200 described above and the differences can be identified by comparing FIG. 2A with FIG. 4A, FIG. 2B with FIG. 4B, and FIG. 2C with FIG. 4C. In figures relating to the article 400, the features that are similar to those shown in figures relating to article 200 are identified with the same reference numeral, incremented by 200. Most of the description of the article 200 of a preferred embodiment above, equally applies to the article 400 and therefore need not be described again in too much detail. Mostly, only the main differences will be discussed.

One main difference is in the shape of the layers all of which are suitably shaped to allow the article 400 to function as a diaper.

Another main difference is the presence of the additional layers, namely a bonding layer 470 and an inner most layer 480 (may also referred as innermost layer). The inner most layer may be in the form of protective strips 480A, 480B that are anti spill strips. The strips 480A, 480B may be nonwoven or may be knitted. The bonding layer 470 is also in the form of strips 470A, 470B. The bonding layer 470 may be positioned or sandwiched between the inner more layer 460 and the inner most layer 480 thereby bonding the inner more layer 460 and the inner most layer 480. This bonding may be by heat and/or adhesive bonding applied in the same or similar manner as already described above. In some embodiments, the outermore layer 420 may also be a stabilising layer (secondary stabilising layer) similar to that in Example 1. In some embodiments, the bonding layer 470 may be made from same material as the stabilising layer 440 and the outermore layer 420 in which case each of the three layers 420, 440 and 480 may function as a stabilising layer to stabilise any nonwoven fibres that are bonded to those layers in the same or similar manner as described above with reference to FIGS. 1A-1D.

The inner more layer 460 may be made from the same textiles as the outer cover 450. I The inner more layer 460 and/or the outer cover 450 may be made from nonwoven textiles or nonwoven material having a plurality of nonwoven fibres. Such nonwoven fibres are stabilised by adhesive and/or heat bonding in a manner that is same or similar as already described above.

Similar to outermore layer 220, the outermore layer 420 may be moisture resistant. In some embodiments, a moisture resistant solution or is applied to the outermore layer 420 The outermore layer 420 may be coated with a moisture resistant solution.

The first and second inner layers 410, 430 may be of same or similar type as already described above for article 200. The outermore layer 420 and the bonding layer 470 may be of same or similar material as the stabilising layer 440. The stabilising layer 440 may be of same material as stabilising layer 240 as described above for article 200. The layers that are described as being optional in article 200 above may also be optional in article 400.

The innermost layer 480 may comprise fastener(s) 467. Similarly, the outer layer 460 may also comprise fastener(s) 468. The fastener(s) 467, 468 may allow releasable coupling with the undergarment in a similar manner as described above for article 200 above. The fastener(s) in this embodiment is most preferably, Velcro® (also known as hook and loop fasteners). However, many other alternative fasteners are possible such as but not limited to buttons, snap domes/poppers etc.

Other than the above-mentioned differences and the differences that are obvious from the figures, all other features of the article 400 including the properties of its various layers may be same as that described above with reference to article 200.

### Example 4

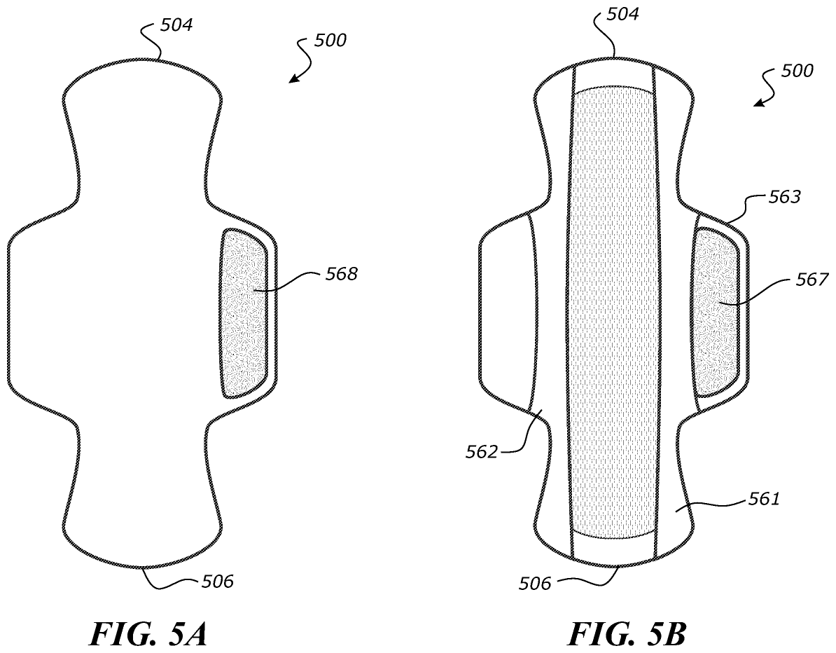

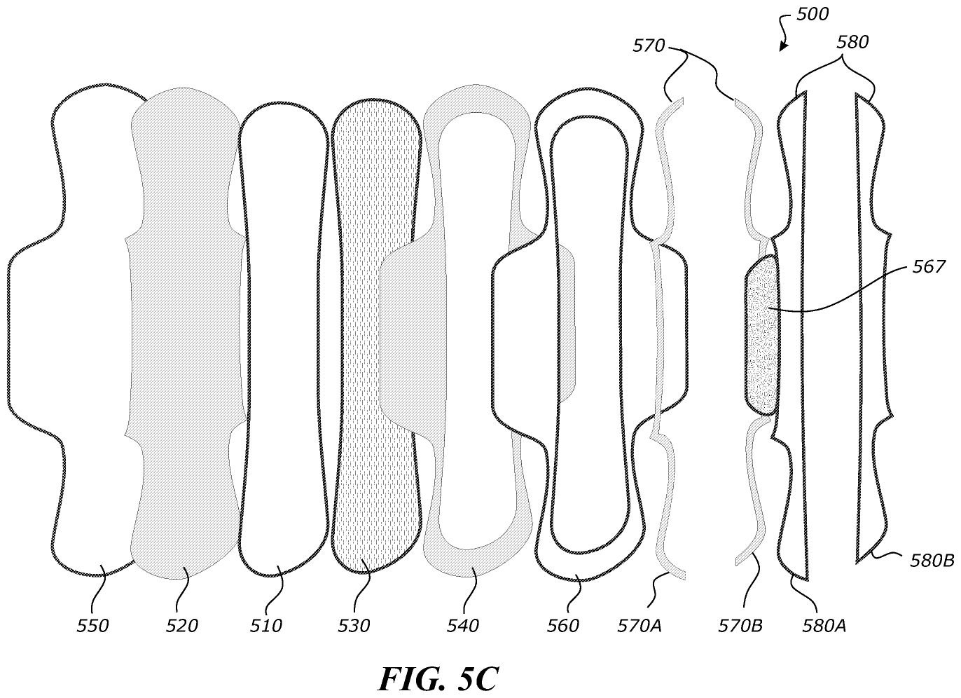

FIGS. 5A-5C show an article 500 according to another preferred embodiment of the present invention.

Article 500 is of or for use with an undergarment to be positioned adjacent a body of a user and to receive moisture from the body of the user. The user may be a person wearing the article. The article 500 may be used as a menstrual fluid absorbing article (also referred to as a “female hygiene article” or “menstrual hygiene article, “female liner” or “menstrual pad”). The article 500 may also be suitable for absorbing any other moisture from the body of the user.

The article 500 is designed for moderate to heavy incontinence.

The article 500 of this example is similar in most aspects to the article 200 described above and the differences can be identified by comparing FIG. 2A with FIG. 5A, FIG. 2B with FIG. 5B, and FIG. 2C with FIG. 5C. In figures relating to the article 500, the features that are similar to those shown in figures relating to article 200 are identified with the same reference numeral, incremented by 300. Most of the description of the article 200 of a preferred embodiment above, equally applies to the article 500 and therefore need not be described again in too much detail. Mostly, only the main differences will be discussed.

One main difference is in the shape of the layers which are of a more suitable shaped to allow the article 500 to be used in situations where incontinence is moderate to heavy. However, outermore layer 520 may be of many other suitable shapes.

Another main difference is the presence of the additional layers, namely bonding layer 570 and an inner most layer 580 (may also referred as innermost layer). The inner most layer 580 are in the form of protective strips 580A, 580B that are anti spill strips. The strips 580A, 580B may be nonwoven or may be knitted. The bonding layer 570 is also in the form of strips 570A, 570B. The bonding layer 570 may be positioned or sandwiched between the inner more layer 560 and the inner most layer 580 thereby bonding the inner more layer 560 and the inner most layer 580. This bonding may be by heat and/or adhesive bonding applied in the same or similar manner as already described above. In some embodiments, the outermore layer 520 may also be a stabilising layer (secondary stabilising layer) similar to that in Example 1. In some embodiments, the bonding layer 570 may be made from same material as the stabilising layer 540 and the outermore layer 520 in which case each of the three layers 520, 540 and 570 may function as a stabilising layer to stabilise any nonwoven fibres that are bonded to those layers in the same manner as described above with reference to FIGS. 1A-1D.

Similar to outermore layer 220, the outermore layer 520 may be moisture resistant. In some embodiments, a moisture resistant solution or is applied to the outermore layer 520 The outermore layer 520 may be coated with a moisture resistant solution.

The inner more layer 560 may be made from the same textiles as the outer cover 550. The inner more layer 560 and/or the outer cover 550 may be made from nonwoven textiles or nonwoven material having a plurality of nonwoven fibres Such nonwoven fibres are stabilised by adhesive and/or heat bonding in a manner that is same or similar as already described above.

The first and second inner layers 510, 530 may be of same or similar type as already described above for article 200. The outermore layer 520 and the bonding layer 570 may be of same or similar material as the stabilising layer 540. The stabilising layer 540 may be of same material as stabilising layer 240 as described above for article 200. The layers that are described as being optional in article 200 above may also be optional in article 500.

The innermost layer 580 may comprise fastener(s) 567. Similarly, the outer layer 560 may also comprise fastener(s) 568. The fastener(s) 567, 568 may allow releasable coupling with the undergarment in a similar manner as described above for article 200 above. The fastener(s) in this embodiment is most preferably, Velcro® (also known as hook and loop fasteners). However, many other alternative fasteners are possible such as but not limited to buttons, snap domes/poppers etc.

Other than the above-mentioned differences and the differences that are obvious from the figures, all other features of the article 500 including the properties of its various layers may be same as that described above with reference to article 200.

### Example 5

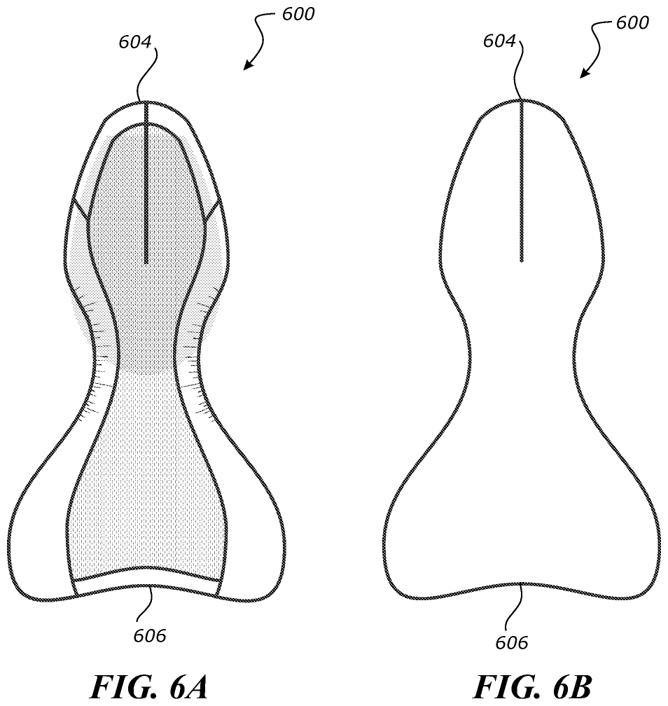

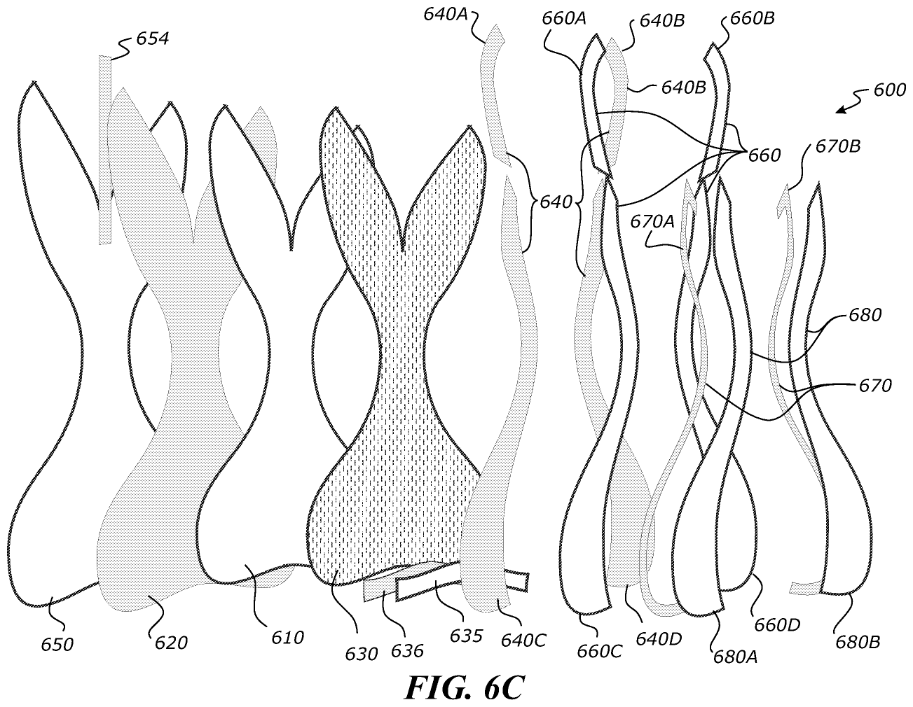

FIGS. 6A-6C show an article 600 according to another preferred embodiment of the present invention.

The article 600 is of or for use with an undergarment to be positioned adjacent a body of a user and to receive moisture from the body of the user. The user may be a person wearing the article. The article 600 may be used as a urine incontinence absorbing and inserting article (also referred to as a “male shield” or “male guard”, or “male pouch” or incontinence pouch”). The purpose is similar to that of article 300 of example 2 above, except article 600 is designed for heavy incontinence. The article 600 may also be suitable for absorbing any other moisture from the body of the user. In some embodiments, the article 600 is an incontinence article for female that is configured to be positioned adjacent a person's genital for receiving menstrual or other fluid from genital area of the person, including urine.

The article 600 of this example is similar in most aspects to the article 300 or article 200 described above and the differences can be identified by comparing FIG. 2A or 3A with FIG. 6B, FIG. 2B or 3B with FIG. 6A, and FIG. 2C or 3C with FIG. 6C. In figures relating to the article 600, the features that are similar to those shown in figures relating to article 200 are identified with the same reference numeral, incremented by 400. Similarly, the features of article 600 that are similar to those shown in figures relating to article 300 are identified with the same reference numeral, incremented by 300. Most of the description of the articles 200 or 300 of a preferred embodiment above, equally applies to the article 600 and therefore need not be described again in too much detail. Only the main differences will be discussed.

One main difference is in the shape of the layers all of which are suitable shaped to function as the urine incontinence absorbing article in heavy continence situation.

Another main difference is the presence of the additional layers, namely a bonding layer 670 and an inner most layer 680 (may also referred as innermost layer). The inner most layer 680 is the form of protective strips 680A, 680B, 680C, 680 D that are anti spill strips. The strips 680A, 680B, 680C, 680D may be nonwoven or may be knitted. The bonding layer 670 is also in the form of strips 670A, 670B. The bonding layer 670 may be positioned or sandwiched between the inner more layer 660 and the inner most layer 680 thereby bonding the inner more layer 660 and the inner most layer 680. This bonding may be by heat and/or adhesive bonding in a manner as already described above. In some embodiments, the outermore layer 620 may also be a stabilising layer (secondary stabilising layer) similar to that in Example 1. In some embodiments, the bonding layer 670 may be made from same material as the stabilising layer 640 and the outermore layer 620 in which case each of the three layers 620, 540 and 670 may function as a stabilising layer to stabilise any nonwoven fibres that are bonded to those layers in the same or similar manner as described above with reference to FIGS. 1A-1D.

The inner more layer 660 may be made from the same textiles as the outer cover 650. The inner more layer 660 and/or the outer cover 650 may be made from nonwoven textiles or nonwoven material having a plurality of nonwoven fibres. Such nonwoven fibres are stabilised by adhesive and/or heat bonding in a manner that is same or similar as already described above.

Similar to outermore layer 220, the outermore layer 620 may be moisture resistant. In some embodiments, a moisture resistant solution or is applied to the outermore layer 620 The outermore layer 620 may be coated with a moisture resistant solution.

Another point of difference is that the stabilisation layer 640 is also in the form of a plurality of strips 640A, 640B, 640C and 640Dto match the shape of the strips 660A, 660B, 660C and 660D of the inner more layer.

Apart from any visual differences, the first and second inner layers 610, 630 may be of same or similar type as already described above for article 200. The layers that are described as being optional in article 200 above may also be optional in article 600. An optional bottom strip 635 may be present as shown in FIG. 6C which is adapted to be bonded with bottom of the surface of the second inner layer 630 that is facing away from the first inner layer 610. The bonding may be by use of a bonding strip 636 configured to be sandwiched between the second inner layer 630 and the bottom strip 635. The bottom strip 635 may be a nonwoven strip in which case the bonding allows stabilisation of the nonwoven fibres of the bottom strip 653. Alternatively, the bottom strip 635 may be a woven or knitted fabric in which case there the fourth stabilising strip 636 may still act as an adhesive strip. Having such arrangement of bottom strip 635 can help avoid the inadvertent transfer of moisture from the bottom of the article. In some embodiments, bonding strip 636 may be made from same material as the stabilising layer 640 to stabilise any nonwoven fibres that are bonded to that strip in the same manner as described above with reference to FIGS. 1A-1D.

Other than the above-mentioned differences and the differences that are obvious from the figures, all other features of the article 500 including the properties of its various layers may be same as that described above with reference to article 200 or 300.

### Example 6

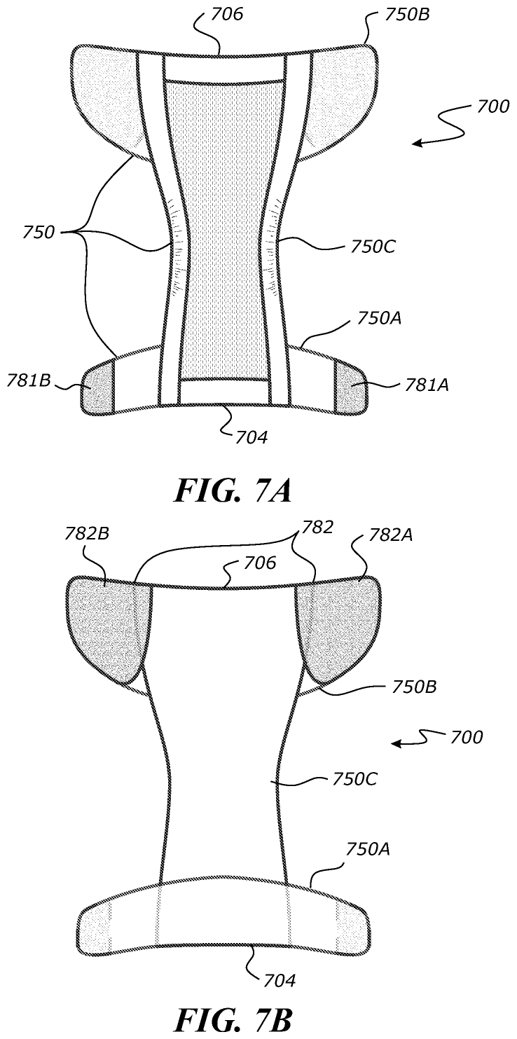

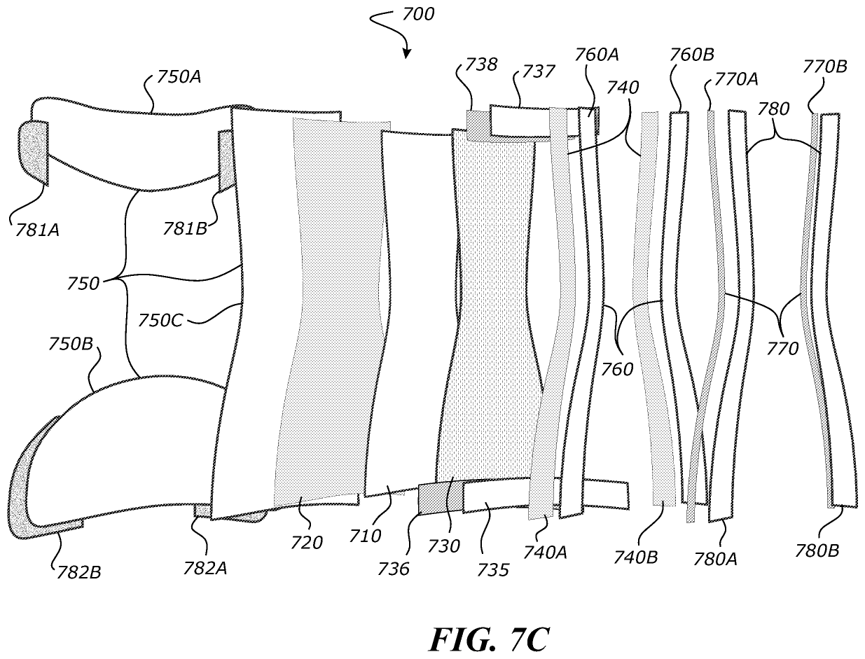

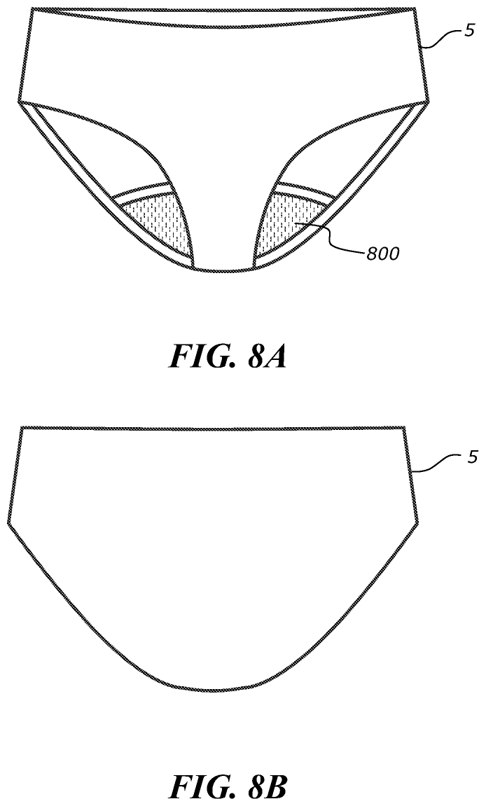

FIGS. 7A-7C show an article 700 according to another preferred embodiment of the present invention. The article 700 in this example is a diaper or pad for adults. It may be a unisex diaper or pad for adults.

The article 700 is of or for use with an undergarment to be positioned adjacent a body of a user and to receive moisture from the body of the user. The user may be an adult person wearing the article.

The article 700 of this example is similar in most aspects to the article 200 described above and the differences can be identified by comparing FIG. 2A with FIG. 7B, FIG. 2B with FIG. 7B, and FIG. 2C with FIG. 7C. In figures relating to the article 700, the features that are similar to those shown in figures relating to article 200 are identified with the same reference numeral, incremented by 500. Most of the description of the article 200 of a preferred embodiment above, equally applies to the article 700 and therefore need not be described again in too much detail. Mostly, only the main differences will be discussed.

One main difference is in the shape of the layers all of which are suitably shaped to allow the article 700 to function as an adult diaper or as pad or unisex diaper or pad for adults.

Another main difference is the presence of the additional layers, namely a bonding layer 770 and an inner most layer 780 (may also referred as innermost layer). The inner most layer may be in the form of protective strips 780A, 780B that are anti spill strips. The strips 780A, 780B may be nonwoven or may be knitted. The bonding layer 470 is also in the form of strips 770A, 770B. The bonding layer 770 may be positioned or sandwiched between the inner more layer 760 and the inner most layer 780 thereby bonding the inner more layer 760 and the inner most layer 780. This bonding may be by heat and/or adhesive bonding in a manner as already described above. In some embodiments, the outermore layer 720 may also be a stabilising layer (secondary stabilising layer) similar to that in Example 1. In some embodiments, the bonding layer 770 may be made from same material as the stabilising layer 740 and the outermore layer 720 in which case each of the three layers 720, 740 and 770 may function as a stabilising layer to stabilise any nonwoven fibres that are bonded to those layers in the same manner as described above with reference to FIGS. 1A-1D.

A further difference is that the inner more layer 760 is also in the form of strips 760A and 760B, each extending from top end to bottom end of the article 700. Additionally, the outermore layer 740 is also in the form of strips 740A and 740B, each extending from top end to bottom end of the article 700.

The inner more layer 760 may be made from the same textiles as the outer cover 750. Inner more layer 760 and/or the outer cover 750 may be made from nonwoven textiles or nonwoven material having a plurality of nonwoven fibres. Such nonwoven fibres are stabilised by adhesive and/or heat bonding in a manner that is same or similar as already described above.

Similar to outermore layer 220, the outermore layer 620 may be moisture resistant. In some embodiments, a moisture resistant solution or is applied to the outermore layer 620 The outermore layer 620 may be coated with a moisture resistant solution.

The first and second inner layers 710, 730 and the stabilising layers may be of same or similar type as already described above for article 200. The layers that are described as being optional in article 200 above may also be optional in article 700.

The outer cover 750 may in the form of a plurality of panels, namely a top panel 750A that is proximal to the top end of the article 700, a bottom panel 750B that is proximal to the bottom end of the article 700 and an intermediate panel 750C extending from the top panel 750A to the bottom panel 750B or vice versa. The intermediate panel 750C may be releasably attached to either or both of the top panel 750A and the bottom panel 750B using a suitable fastener(s) 781A, 781B. The fastener(s) in this embodiment is most preferably, Velcro® (also known as hook and loop fasteners). However, many other alternative fasteners are possible such as but not limited to buttons, snap domes/poppers etc.

The outer facing surface of the outer cover 750 may comprise fastener(s) 782A, 782B. The fastener(s) 782A, 782B may allow releasable coupling with the undergarment in a similar manner as described above for article 200 above. The fastener(s) in this embodiment is most preferably, Velcro® (also known as hook and loop fasteners). However, many other alternative fasteners are possible such as but not limited to buttons, snap domes/poppers etc.

An optional bottom strip 735 may be present as shown in FIG. 7C which is adapted to be bonded with bottom of the surface of the second inner layer 730 that is facing away from the first inner layer 710. The bonding may be by use of a bottom bonding strip 736 which is configured to be sandwiched between the second inner layer 730 and the bottom strip 635. The bottom strip 635 may be a nonwoven strip in which case the bonding allows stabilisation of the nonwoven fibres of the bottom strip. Alternatively, the bottom strip 735 may be a woven or knitted fabric in which case the bottom bonding strip 736 may still act as an adhesive strip. Having such arrangement of bottom strip 735 can help avoid the inadvertent transfer of moisture from the bottom of the article 700. In some embodiments, bottom bonding strip 736 may be made from same material as the stabilising layer 740 to stabilise any nonwoven fibres that are bonded to that strip in the same manner as described above with reference to FIGS. 1A-1D.

Similarly optional top strip 737 may be present as shown in FIG. 7C which is adapted to be bonded with top of the surface of the second inner layer 730 that is facing away from the first inner layer 710. The bonding may be by use of a top bonding strip 738 which is configured to be sandwiched between the second inner layer 730 and the top strip 737. The top strip 635 may be a nonwoven strip in which case the bonding allows stabilisation of the nonwoven fibres of the bottom strip. Alternatively, the top strip 735 may be a woven or knitted fabric in which case there the top bonding strip 738 may still act as an adhesive strip. Having such arrangement of top strip 737 can help avoid the inadvertent transfer of moisture from the top of the article 700. In some embodiments, bottom top bonding strip 738 may be made from same material as the stabilising layer 740 to stabilise any nonwoven fibres that are bonded to that strip in the same manner as described above with reference to FIGS. 1A-1D.

Other than the above-mentioned differences and the differences that are obvious from the figures, all other features of the article 700 including the properties of its various layers may be same as that described above with reference to article 200.

### Example 7

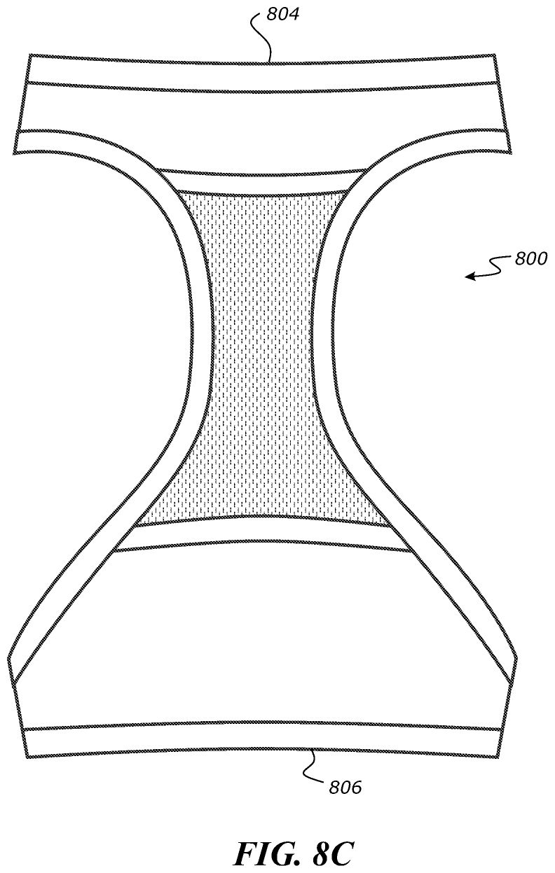

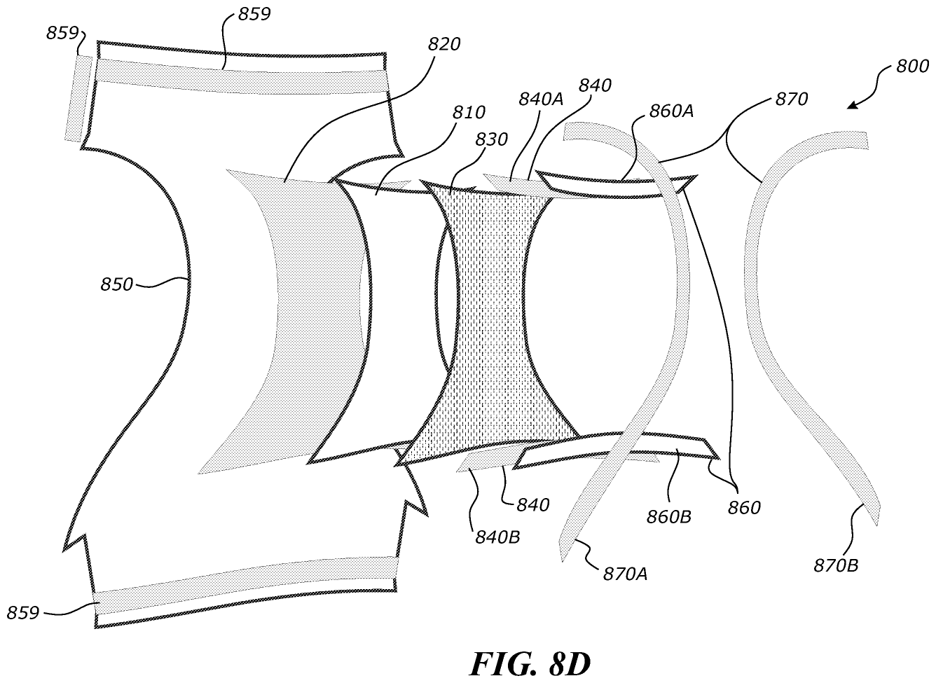

FIGS. 8A-8D show an article 800 according to another preferred embodiment of the present invention. The article 200 in this example is a female underwear 5 or a part of the female underwear 5.

The article 800 of this example is similar in most aspects to the article 200 described above and the differences can be identified by comparing FIG. 2A with FIG. 8C and FIG. 2B with FIG. 8D. In FIGS. 8A-8D, the features that are similar to those shown in figures relating to article 200 are identified with the same reference numeral, incremented by 600. Most of the description of the article 200 of a preferred embodiment above, equally applies to the article 800 and therefore need not be described again in too much detail. Mostly, only the main differences will be discussed.

Here the outer cover 850 is a female underwear panel and may be made from knitted textiles and are preferably highly stretchable for comfort.

The outermore layer 820 is positioned or sandwiched between the first inner layer 810 and the outer cover 850 thereby interconnecting the first inner layer 810 and the outer cover 850.

The first inner layer 810 and the outer cover 860 are bonded together using the outermore layer 820.

The inner more layer 860 is in the form of strips 860A, 860B. A stabilising layer 840 is also in the form of strips 840A, 840B. Strip 840A may be positioned or sandwiched between the second inner layer 830 and the inner more layer strip 860A. Similarly, strip 840B may be positioned or sandwiched between the second inner layer 830 and the inner more layer strip 860B. Therefore, the stabilising strips 840A, 840B may interconnect the second inner layer 830 and the inner more strips 860A, 860B.

The inner more strips 860A, 860B may be made from the same textiles as the outer cover 850. Alternatively, the inner more strips may be made from nonwoven textiles.

The inner more strips 860A, 860B and the stabilising strips 840A, 840B may laterally extend along a first edge of the second inner layer 830 that is proximal to the top end 804 of the article 800 and a second edge that is proximal to the bottom end 806 of the article 800. More specifically, the inner more strip 860A and the stabilising strip 840A may laterally extend along a first edge of the second inner layer 830 that is proximal to the top end 804 of the article 800. Similarly, the inner more strip 860B and the stabilising strip 840B may laterally extend along a second edge of the second inner layer 860B that is proximal to the bottom end 806 of the article 800.

Bonding layer 870 may be in the form of two strips 870A, 870B may be present. The third stabilising strips 870A, 870B may be adhesive strips. The bonding strips 870A, 870B may be bonded (adhesively and/or heat bonded) to two longitudinal sides of the second inner layer 830 and also to the inner more strips 860A and 860B. Further adhesive strips 859 are shown which may optionally be of similar material as stabilising layer 840. In some embodiments, the outermore layer 820 may also be a stabilising layer (secondary stabilising layer) similar to that in Example 1. In some embodiments, the bonding layer 870 may be made from same material as the stabilising layer 840 and the outermore layer 820 in which case each of the three layers 820, 840 and 870 may function as a stabilising layer to stabilise any nonwoven fibres that are bonded to those layers in the same manner as described above with reference to FIGS. 1A-1D.

Other than the above-mentioned differences and any other differences that are obvious from the figures, all other features of the article 800 including the properties of its various layers may be same as that described above with reference to article 200.

### Example 8

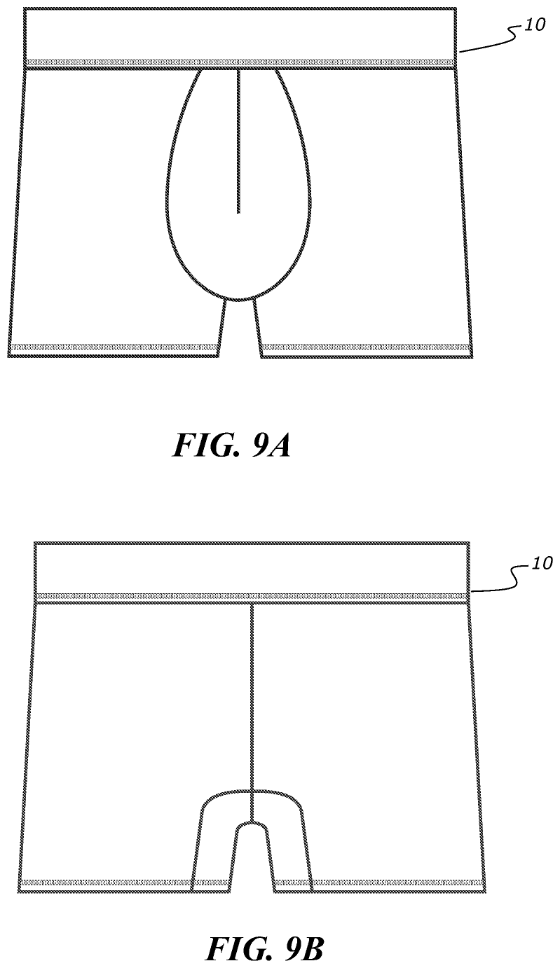

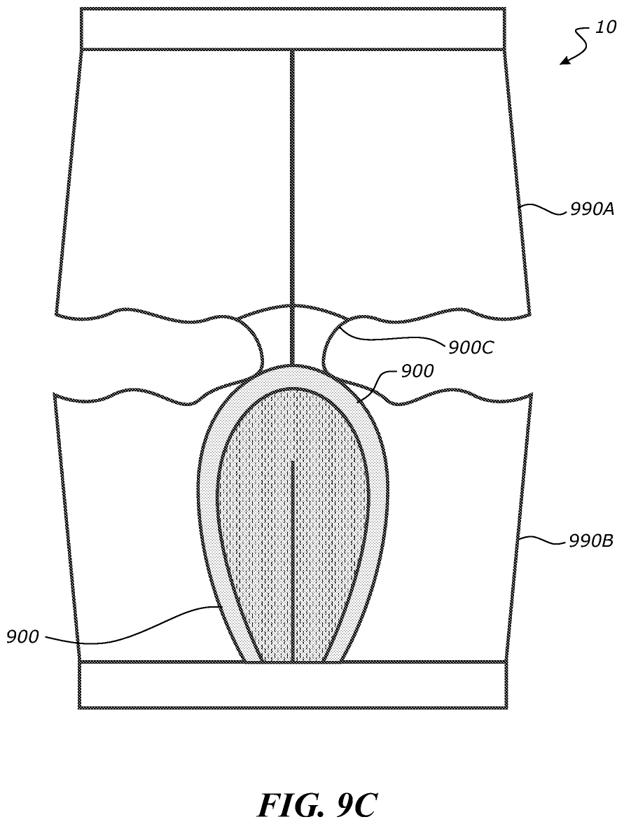

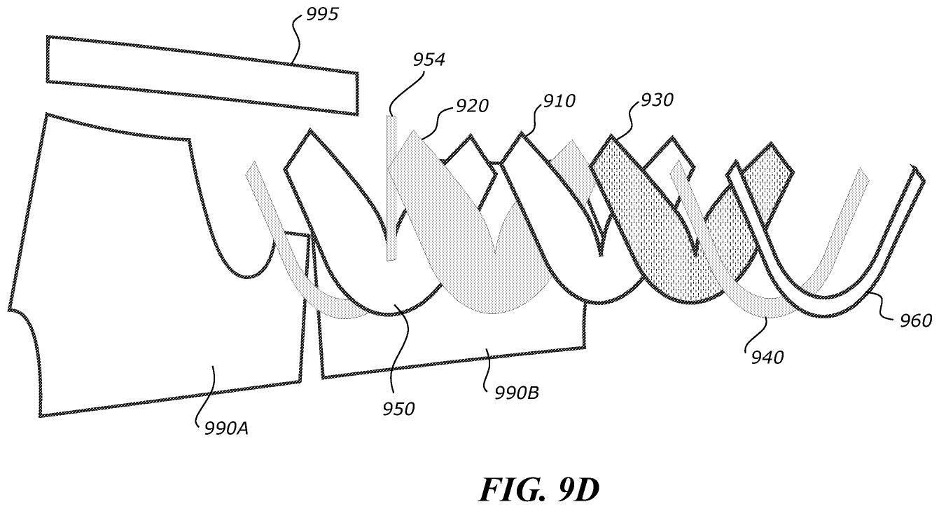

FIGS. 9A-9B are front and rear views of a men's/male's underwear 10 comprising the article 900 according to a further embodiment of the present invention that is same or similar to the article 300 of example 2.

The article 900 of this example is similar in most aspects to the article 300 described above and the differences can be identified by comparing FIG. 9D with FIG. 3C. In figures relating to the article 900, the features that are similar to those shown in figures relating to article 300 are identified with the same reference numeral, incremented by 600. Most of the description of the article 300 of a preferred embodiment above, equally applies to the article 900 and therefore need not be described again in too much detail. Mostly, only the main differences will be discussed.

FIG. 9C shows a flattened view of the underwear 10 showing the article 900 forming part of male underwear. FIG. 12D shows an exploded view showing the article 900 forming part of male underwear. The outer cover 950 may be coupled to a male underwear panel 990A and may be made from knitted textiles and are preferably highly stretchable for comfort. Alternatively, the outer over 950 may be made from nonwoven fibres similar to outer cover 450. The other panels 990A, 990B, 990C of the underwear are also shown. These panels may also be made from knitted textiles and are preferably highly stretchable. A waist elastic 995 of the underwear is also shown. These parts of underwear may be pre-coupled with the underwear panel using adhesive strip 960 (which may be similar to the stabilising layer 940) either prior or after the bonding of the various layers of the article 900 using compressive force.

Other than the above-mentioned differences and any other differences that are obvious from the figures, all other features of the article 900 including the properties of its various layers may be same as that described above with reference to article 300.

### Example 9

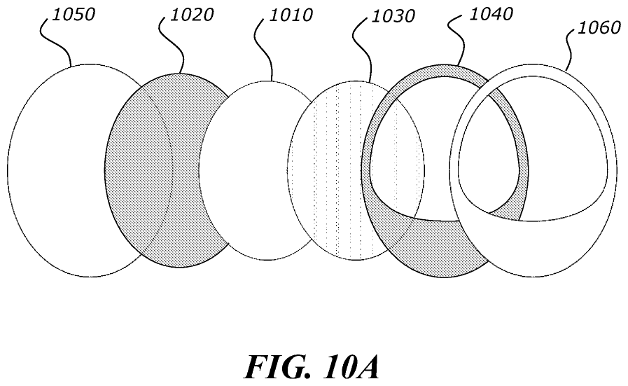

FIG. 10A shows an exploded view of an article 1000 according to another preferred embodiment of the present invention.

The article 1000 is of or for use with an undergarment to be positioned adjacent a breast of a user and to receive moisture from the breast of the user. The article 1000 may be used as a nursing pad. The article 1000 may also be suitable for absorbing any other moisture from the body of the user.

The article 1000 of this example is similar in most aspects to the article 200 described above and the differences can be identified by comparing FIG. 2C with FIG. 10A. In figure relating to the article 1000, the features that are similar to those shown in figures relating to article 200 are identified with the same reference numeral, incremented by 800. Most of the description of the article 200 of a preferred embodiment above, equally applies to the article 1000 and therefore need not be described again in too much detail. Mostly, only the main differences will be discussed.

One main difference is in the shape of the layers all of which are suitable shaped to function as the nursing pad.

Similar to outermore layer 220, the outermore layer 1020 may be moisture resistant. In some embodiments, a moisture resistant solution or is applied to the outermore layer 1020 The outermore layer 1020 may be coated with a moisture resistant solution.

The inner more layer 1060 may be made from the same textiles as the outer cover 1050. The inner more layer 1060 and/or the outer cover 1050 may be made from nonwoven textiles or nonwoven material having a plurality of nonwoven fibres. Such nonwoven fibres are stabilised by adhesive and/or heat bonding in a manner that is same or similar as already described above.

The first and second inner layers 1010, 1030 may be of same or similar type as already described above for article 200. The outermore layer 1020 may be of same or similar material as the stabilising layer 1040. The stabilising layer 1040 may be of same material as stabilising layer 240 as described above for article 200. Any layers that are described as being optional in article 200 above may also be optional in article 1000.

Other than the above-mentioned differences and any other differences that are obvious from the figures, all other features of the article 1000 including the properties of its various layers may be same as that described above with reference to article 200.

### Example 10

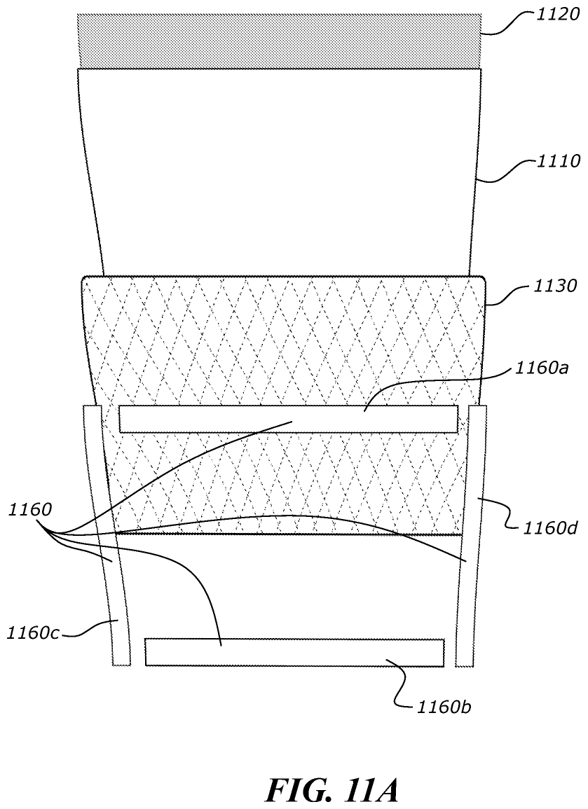

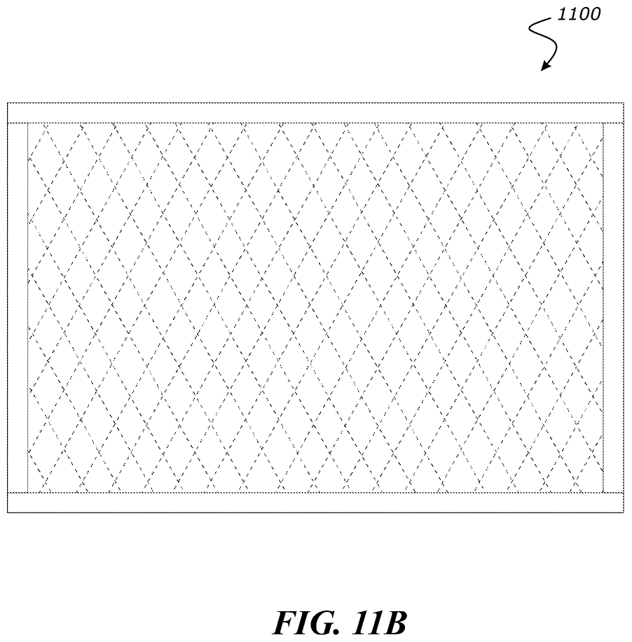

FIG. 11A shows an exploded view of an article 1100 according to another preferred embodiment of the present invention. FIG. 11B shows an assembled view of the article 1100. The article 1100 is of or for use as a sanitary protector or underpad or incontinence pad. Some non-limiting examples of article 1100 may be underpad for bed, underpad for seat, car seat protector, baby diaper changing pad, baby cot protector or for protection from bladder leaks/incontinence.

The article 1100 of this example is similar in most aspects to the article 200 described above and the differences can be identified by comparing FIG. 2C with FIG. 11A. In figure relating to the article 1100, the features that are similar to those shown in figures relating to article 200 are identified with the same reference numeral, incremented by 900. Most of the description of the article 200 of a preferred embodiment above, equally applies to the article 1100 and therefore need not be described again in too much detail. Mostly, only the main differences will be discussed.

One main difference is in the shape of the layers all of which are suitable shaped to function as a sanitary protector or underpad or incontinence pad.

Similar to outermore layer 220, the outermore layer 1120 may be moisture resistant. In some embodiments, a moisture resistant solution or is applied to the outermore layer 1120. The outermore layer 1120 may be coated with a moisture resistant solution. In this example, it can be seen that the outermore layer 1120 is the outermost layer of the article 1100. In some embodiments, a separate outer cover may be present as the outermost layer adjacent the outermore layer 1120. In such a case, outer cover may be nonwoven material similar to outer cover 250.

The inner more layer 1160 may be made from nonwoven textiles or nonwoven material having a plurality of nonwoven fibres. Such nonwoven fibres are stabilised by adhesive (e.g., bonding glue) and/or heat bonding in a manner that is same or similar as already described above. The inner more layer 1060 may be in the form of strips as shown in FIG. 11A.

The inner layer 1110 may be of same or similar type as already described above for inner layer 210 for article 200. Although only one inner layer 1110 in the form of non-woven absorbent core is disclosed, there may be two or more such non-woven layers. The outermore layer 1020 may be of same or similar material as the stabilising layer 1040. Also, disclosed is a second inner layer 1130. The second inner layer may be a quick dry knit textile. The second inner layer may be a quick dry knit textile with quilt stich to reduce friction.

Other than the above-mentioned differences and any other differences that are obvious from the figures, all other features of the article 1000 including the properties of its various layers may be same as that described above with reference to article 200.

It will of course be realised that while the foregoing has been given by way of illustrative example of the present invention, all such modifications and variations thereto as would be apparent to a person skilled in the art are deemed to fall within the broad scope and ambit of the various aspects of invention as is hereinbefore described and/or defined in the claims.

From the above, it can be appreciated that the present invention allows the nonwoven fibres to be bonded thereby stabilising the nonwoven fibres. The stabilisation of nonwoven fires means, the fibres will not or will not easily disintegrate during washing thereby allowing the otherwise nonreusable nonwoven fibres to become reusable. Also, with the present invention layers of the article to be bonded together by simply arranging the layers as described above applying a compressive force on the article. This eliminates or at least reduces stitching and/or other complex process for bonding the layers together thereby significantly reducing the manufacturing process, costs and time. Also, complex machinery will note be required for manufacturing the article. Also, no or less stitching means the article is less prone to leakage. In some embodiments, the article is leakproof.

One or more non-limiting advantages of the present invention may be as follows:
Simpler design
Cost effective
Easy to assemble
No complex machinery is required for assembly of layers as the layers can be bonded simply by applying compressive force on the article-i.e., pressing.
Reduction in Manufacturing Time
Less labour intensive to manufacture/assemble as no complex bonding process
Reusable article-that is environmentally friendlier than single-use products
Free of any harmful or hazardous chemicals
Can be washed and tumbled dried.
Less prone to leakages due to less or no stitching
Absorbency speed, levels & liquid retention abilities are not compromised whist significantly reducing the number of steps, time, and complexity of the assembly of layers during manufacturing.

1. A method of stabilising nonwoven fibres, the method comprising the steps of:
providing a nonwoven material comprising or in the form of a plurality of nonwoven fibres;
providing a stabilising material;
applying the stabilising material to at least a portion or all of the nonwoven material so that the stabilising material is applied to at least some or all of the plurality of nonwoven fibres, the stabilising material being a material that is configured to bond the nonwoven fibres by adhesive and/or heat bonding; and
bonding the plurality of nonwoven fibres to which the stabilising material is applied with the stabilising material by adhesive and/or heat bonding.

2. The method of claim 1, wherein the nonwoven fibres are bonded to form a bonded nonwoven fibre layer comprising at least a portion of nonwoven fibres that are bonded to the extent that the bonded nonwoven fibres do not disintegrate or substantially disintegrate when the layer is washed or laundered at least once or multiple times.

3. The method of claim 1 or 2, wherein the nonwoven material is nonwoven material layer.

4. The method of claim 1 or 2, wherein the nonwoven material is a nonwoven a nonwoven fabric or a nonwoven textile.

5. The method of any one of claim 1 to 4, wherein the heat bonding comprises fusing at least some or all of the nonwoven fibres at a temperature that correlates to a melting point of the fibres that are to be fused and/or a melting point of the stabilising material.

6. The method of any one of claim 1 to 5, wherein the heat bonding occurs at a temperature of between 70 degrees Celsius and 240 degrees Celsius.

7. The method of any one of claims 1 to 6, wherein the stabilising material is formed as a stabilising layer.

8. The method of claim 7, wherein the stabilising layer is a membrane or a film.

9. The method of claim 7 or 8, wherein the stabilising layer has a thickness based on a type of the nonwoven material and the melting point of the nonwoven material.

10. The method of any one of claims 7 to 9, wherein the stabilising layer has a thickness between 0.005 mm and 0.5 mm.

11. The method of any one of claims 7 to 10, wherein the stabilising layer is a breathable layer.

12. The method of any one of claims 7 to 11, wherein the stabilising layer is a full layer.

13. The method of any one of claims 7 to 11, wherein the stabilising layer is a partial layer.

14. The method of any one of claims 7 to 11, wherein stabilising layer is a framed layer.

15. The method of any one of claims 7 to 14, wherein stabilising layer is a polyurethane (PU) material layer.

16. The method of any one of claims 7 to 14, wherein the stabilising layer is a polyethylene (PE) material layer.

17. The method of any one of claims 7 to 14, wherein the stabilising layer is a polylactic acid (PLA) material layer.

18. The method of any one of claims 7 to 17, wherein the stabilising layer is a textile adhesive layer.

19. The method of any one of claims 1 to 18, wherein bonding occurs in a patterned manner.

20. The method of any one of claims 1 to 19, wherein the stabilising material is an adhesive material.

21. The method of claim 20, wherein the adhesive material is a glue.

22. The method of claims 20 or 21, wherein the adhesive material is a spray or a roll-on glue.

23. The method of claim 20, wherein the adhesive material is a polyurethane (PU) adhesive.

24. The method of claim 20, wherein the adhesive material is a polyethylene (PE) adhesive.

25. The method of claim 20, wherein the adhesive material is a polylactic acid (PLA) adhesive.

26. The method of claim 20, wherein the adhesive material is a textile adhesive.

27. The method of claims 1 to 26, wherein an adhesive substance is applied to the stabilising material for the bonding.

28. The method of claim 20, wherein the adhesive substance is a glue, a spray glue, a roll-on glue, a polyurethane adhesive, a polyethylene (PE) adhesive, or a polylactic acid (PLA) adhesive.

29. The method of claim 20, wherein the adhesive substance is a textile adhesive.

30. The method of any one of claims 1 to 29, wherein the nonwoven fibres are synthetic polymers, biocomponent fibres, natural fibres, manmade fibres or a combination thereof.

31. The method of claim 30, wherein the synthetic polymers are selected from polyethylene (PE), polypropylene (PP) or polyethylene terephthalate (PET).

32. The method of claim 30, wherein the natural fibres are selected from wood, pulp, cotton or bamboo.

33. The method of claim 30, wherein the manmade fibres are selected from Viscose, Lyocell, polylactic acid (PLA) or cellulose acetate.

34. A method of constructing a reusable moisture absorbing article of or for use with an undergarment to be positioned adjacent a body of a user and to absorb (preferably, absorb and retain) body moisture from the user, the method comprising:
providing at least one a layer of nonwoven material comprising or in the form of a plurality of non-woven fibres;
applying at least one stabilising material to at least a portion of the at least one layer of the nonwoven material so that the stabilising material is applied to at least some or all of the plurality of nonwoven fibres, the stabilising material being a material that is configured to bond the nonwoven fibres by adhesive and/or heat bonding and the stabilising material forming a stabilising layer; and
bonding the plurality of nonwoven fibres to which the stabilising material is applied with the stabilising material by adhesive and/or heat bonding, thereby forming the reusable moisture absorbing article.

35. The method of claim 34, wherein the nonwoven fibres are bonded to the extent that the bonded nonwoven fibres do not disintegrate or substantially disintegrate when exposed to washing or laundering at least once or multiple times.

36. A method of constructing a reusable moisture retaining article of or for use with an undergarment to be positioned adjacent a body of a user and to absorb and retain body moisture from the user, the method comprising:
providing a reusable moisture absorbing and retaining insert at least a portion of which is exposed or configured to be exposed to define an inner layer of the article, the inner layer being configured to be located adjacent the user's body and facing towards the user's body for being in bodily contact with the user to directly absorb and retain the body moisture from the user, the insert comprising at least one inner layer of a nonwoven material comprising or in the form of a plurality of non-woven fibres;
providing an inner more layer of a nonwoven material comprising or in the form of a plurality of nonwoven fibres located outwardly of the insert;
providing a stabilising layer formed by a stabilising material, the stabilising material being a material that is configured to bond the nonwoven fibres by adhesive and/or heat bonding;
providing an outer cover that is configured to define an outer layer of the article; and
assembling the inset, the inner more layer, the stabilising layer, the outer cover by:
positioning the inner more layer, the insert, the stabilising layer and the outer cover so that the insert is located between the outer cover and the stabilising layer, at least a first portion of the stabilising layer is located between the inner more layer and the insert, wherein at least a second portion of the stabilising layer is also configured to bond with the outer cover,
bonding at least a portion of the inner more layer with the stabilising layer by applying heat and/or adhesive bonding,
bonding at least a portion of the insert with the stabilising layer by applying the heat and/or adhesive bonding, and
bonding at least a portion of the outer cover with the second portion of the stabilising layer by applying the heat and/or adhesive bonding, the insert being located between the outer cover and the stabilising layer.

37. A reusable moisture absorbing article comprising a plurality of the nonwoven fibres that are stabilised using the method as claimed in any one of claims 1 to 36.

38. The article of claim 37, wherein the article is any one of the following:
a diaper,
a sanitary napkin,
an incontinence pad,
an incontinence pouch,
a female underwear for incontinence,
a male underwear for incontinence,
a nursing pad,
an underpad,
a baby diaper changing pad, or
a baby cot protector.

39. A reusable moisture absorbing article manufactured using a method as claimed in any one of claims 1 to 36.

40. A reusable moisture absorbing article, the article comprising:
at least one nonwoven material comprising or in the form of a plurality of nonwoven fibres;
at least one stabilising material;
wherein at least one nonwoven layer and at least one stabilising layer are bonded together by applying the stabilising material to at least a portion or all of the nonwoven material so that the stabilising material is applied to at least some or all of the plurality of nonwoven fibres; and
bonding the plurality of nonwoven fibres to which the stabilising material is applied with the stabilising material by adhesive and/or heat bonding.

41. An undergarment that comprises or incorporates an article that is constructed using any of the method as claimed in any one of claim 1 to 36.
